(19) United States
(12) Patent Application Publication
(10) Pub. No.: US 2025/0221947 A1
(43) Pub. Date: Jul. 10, 2025

(54) METHODS FOR TREATING A CORONAVIRUS INFECTION USING VALPROIC ACID AND DOCOSAHEXAENOIC ACID

(71) Applicant: Board of Regents, The University of Texas System, AUSTIN, TX (US)

(72) Inventors: RONALD RODRIGUEZ, San Antonio, TX (US); WASIM H. CHOWDHURY, San Antonio, TX (US); DEAN BACICH, San Antonio, TX (US)

(21) Appl. No.: 19/015,438

(22) Filed: Jan. 9, 2025

Related U.S. Application Data
(60) Provisional application No. 63/619,137, filed on Jan. 9, 2024.

(51) Int. Cl. A61K 31/19; A61K 31/202; A61K 38/15; A61K 45/06; A61P 31/14

(52) U.S. Cl. CPC A61K 31/19; A61K 31/202; A61K 38/15; A61K 45/06; A61P 31/14

(57) ABSTRACT

Disclosed herein, are methods for treating, preventing or inhibiting a coronavirus infection or a disease or condition associated with a coronavirus infection. The methods can comprise administering to the subject one or more therapeutically effective doses of valproic acid and docosahexaenoic acid.

### CROSS REFERENCE TO RELATED APPLICATIONS

This application claims the benefit of the filing date of U.S. Provisional Application No. 63/619,137, filed on Jan. 9, 2024. The content of this earlier filed application is hereby incorporated by reference in its entirety.

### REFERENCE TO A SEQUENCE LISTING

The Sequence Listing submitted herein as a text file named “21105_0087U2_SL.xml,” created on Jan. 9, 2025, and having a size of 4,096 bytes is hereby incorporated by reference.

### BACKGROUND

The SARS-CoV-2 pandemic has resulted in unprecedented worldwide infections, with the continuing development of mutant variants of varying degrees of infectivity and virulence (da Silva, S. J. R., et al., Two Years into the COVID-19 Pandemic: Lessons Learned. ACS Infect Dis, 2022). The inability to implement a worldwide persistently effective, highly penetrant vaccination strategy suggests that timely eradication of this viral pathogen is unlikely. Even with the advent of new antiviral agents, the disease burden worldwide continues to exceed current preventative and therapeutic strategies. Developing affordable, broadly effective antiviral therapeutics is needed.

### SUMMARY

Disclosed herein are methods treating a subject having a coronavirus infection, the methods comprising administering to the subject one or more therapeutically effective doses of valproic acid and docosahexaenoic acid.

Disclosed herein are methods of treating or preventing COVID-19 in a subject, the methods comprising administering to the subject one or more therapeutically effective doses of valproic acid and docosahexaenoic acid.

Disclosed herein are methods of preventing or inhibiting a coronavirus infection in a subject, the methods comprising administering to the subject one or more therapeutically effective doses of valproic acid and docosahexaenoic acid.

Disclosed herein are methods of inhibiting replication of a coronavirus in a subject having a coronavirus infection, the methods comprising administering to the subject one or more therapeutically effective doses of valproic acid and docosahexaenoic acid.

Disclosed herein are methods of reducing one or more symptoms of a coronavirus infection or COVID-19 in a subject, the methods comprising administering to the subject one or more therapeutically effective doses of valproic acid and docosahexaenoic acid.

Disclosed herein are methods of inhibiting replication of a coronavirus in a cell, the methods comprising contacting the cell infected with the coronavirus with one or more therapeutically effective doses of valproic acid and docosahexaenoic acid.

Disclosed herein are methods of inhibiting, treating or preventing a coronavirus infection in a subject, the methods comprising administering to the subject having said infection a plurality of therapeutically effective doses of valproic acid and docosahexaenoic acid.

Disclosed herein are kits for use in treating a subject suffering from a coronavirus infection, said kits comprising: (a) valproic acid; and (b) docosahexaenoic acid.

Disclosed herein are kits for use in treating a subject suffering from a coronavirus infection, said kit comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, risperdal, differin, plitidepsin, convalescent plasma, actemra, recombinant soluble ACE2, camostate mesylate and analogs thereof, fluvoxamine, or prednisone.

Disclosed herein are kits for use in treating a subject suffering from a coronavirus infection, said kit comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

Disclosed herein are kits for use in preventing or inhibiting a coronavirus infection in a subject, said kits comprising: (a) valproic acid; and (b) docosahexaenoic acid.

Disclosed herein are kits for use in preventing or inhibiting a coronavirus infection in a subject, said kits comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone.

Disclosed herein are kits for use in preventing or inhibiting a coronavirus infection in a subject, said kits comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

Disclosed herein are kits for use in inhibiting replication of a coronavirus infection in a subject, said kits comprising: (a) valproic acid; and (b) docosahexaenoic acid.

Disclosed herein are kits for use in inhibiting replication of a coronavirus infection in a subject, said kits comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone.

Disclosed herein are kits for use in inhibiting replication of a coronavirus infection in a subject, said kits comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

Disclosed herein are methods of treating a subject having a coronavirus infection, the methods comprising administering to the subject one or more therapeutically effective doses of (a) depsipeptide and (b) docosahexaenoic acid.

Disclosed herein are methods of treating or preventing COVID-19 in a subject, the methods comprising administering to the subject one or more therapeutically effective doses of (a) depsipeptide and (b) docosahexaenoic acid.

Disclosed herein are methods of preventing or inhibiting a coronavirus infection in a subject, the methods comprising administering to the subject one or more therapeutically effective doses of (a) depsipeptide and (b) docosahexaenoic acid.

Disclosed herein are methods of inhibiting replication of a coronavirus in a subject having a coronavirus infection, the methods comprising administering to the subject one or more therapeutically effective doses of (a) depsipeptide and (b) docosahexaenoic acid.

Disclosed herein are methods of reducing one or more symptoms of a coronavirus infection or COVID-19 in a subject, the methods comprising administering to the subject one or more therapeutically effective doses of (a) depsipeptide and (b) docosahexaenoic acid.

Disclosed herein are methods of inhibiting replication of a coronavirus in a cell, the method comprising contacting the cell infected with the coronavirus with one or more therapeutically effective doses of (a) depsipeptide and (b) docosahexaenoic acid.

Disclosed herein are methods of inhibiting, treating or preventing a coronavirus infection in a subject, the methods comprising administering to the subject having said infection a plurality of therapeutically effective doses of (a) depsipeptide and (b) docosahexaenoic acid.

### BRIEF DESCRIPTION OF THE DRAWINGS

FIGS. 1A-D show viral inhibition assays around the IC50 of VPA by direct inhibition of HDAC2. FIG. 1A shows antibody-based assay infected with SARS-CoV2. FIG. 1B shows firefly luciferase assay infected with SARS-CoV2. FIG. 1C shows oligonucleotide hybridization-based assay infected with HCoV-229E. FIG. FIG. 1D shows oligonucleotide hybridization-based assay infected with HCoV-229E with/without VPA preincubation.

FIGS. 2A-D show host cellular genes interactions with the SARS-CoV2 proteins. FIG. 2A show volcano plot for gene expression levels of selected 300 Gordon gene set at 24 hours (left) and 48 hours (right). FIG. 2B shows virus-host protein interaction map of the Gordon gene sets that met significance criteria for viral assembly, replication and pathogenicity. FIG. 2C shows protein levels measured by Western blot for selected gene sets PCNT, DNMT1, BRD2, and HMOX1 at 24 hours (top) and 48 hours (bottom). FIG. 2D shows differentially expressed genes upon VPA treatment for 24, 48, 72 and 96 hours.

FIGS. 3A-D show the effect of different polyunsaturated fatty acids (PUFAs) in the inhibition of HCoV-229E viral replication in MRC5 cells using antibody-based assay demonstrates significant inhibition of viral replication. FIG. 3A shows the IC50 value of linoleic acid (LA), demonstrating significant inhibition of viral replication. FIG. 3B shows the IC50 value of docosahexaenoic acid (DHA), demonstrating significant inhibition of viral replication.

FIG. 3D shows the IC50 value of eicosapentaenoic acid (EPA), demonstrating significant inhibition of viral replication. FIG. 4D shows the IC50 value of alpha-linolenic acid (ALA), demonstrating least inhibition.

FIGS. 4A-D show viral inhibition assay in combination of VPA with or without fixed dose (25 μM) of both DHA and LA. FIG. 4A shows the IC50 value of VPA with or without linoleic acid (LA). FIG. 4B shows the IC50 value of VPA with or without docosahexaenoic acid (DHA). FIG. 4C shows the IC50 value of trichostatin A (TSA) with or without docosahexaenoic acid (DHA). FIG. 4D shows the IC50 value of depsipeptide (Dep) with or without docosahexaenoic acid (DHA).

FIGS. 5A-E show the impact of VPA and DHA in gene expression patterns of MRC5 cells when infected with coronavirus. FIG. 5A shows the percentage of viral RNA sequence detected in RNA-seq from total RNA content of MRC5 cells infected with HCoV-229E at different experimental conditions and different drug combinations. FIG. 5B shows the number of differentially expressed genes (DEGs) identified in MRC5 cells when treated with different drug combinations. MRC5 cells, drug-alone compared to mock-treated (left). MRC5 cells infected with HCoV-229E treated with drug compared to MRC5 cells infected with HCoV-229E alone (right). FIG. 5C shows K-means clustering scatterplot comparing the Log₂(fold change) expression for each treatment group (VPA, DHA, VPA+DHA) versus the MRC5 control cells.

FIG. 5D shows K-means clustering scatterplot comparing the Log₂ (fold change) expression for each treatment group (VPA, DHA, VPA+DHA) versus HCoV-229E infected MRC5 cells. FIG. 5E shows principle component analysis (PCA) of the differential gene expression obtained under different treatment conditions. These data in FIGS. 5C and 5D demonstrate how the combination of VPA and DHA result in the global shift of 2000 genes from proviral replication to antiviral replication as the group of genes had a net positive slope in expression level to net negative slope when the cells were incubated with both VPA and DHA when infected with coronavirus. This fundamental shift occurs because of unique mechanism of action affecting many multiple pathways, such that signal point mutations in the virus are unlikely to confer resistance to the combined effects of VPA+DHA on viral inhibition.

FIGS. 6A-H show scatter plots of the molecular pathways obtained from ingenuity pathway analysis (IPA) under different treatment combinations of DHA and VPA. MRC5 cells were treated with (FIG. 6A) 25 μM DHA, (FIG. 6B) 25 μM DHA and infected with HCoV-229E, (FIG. 6C) 0.5 mM VPA, (FIG. 6D) 0.5 mM VPA and infected with HCoV-229E, (FIG. 6E) 25 μM DHA+0.5 mM VPA, (FIG. 6F) 25 μM DHA+0.5 mM VPA and infected with HCoV-229E, (FIG. 6G) 25 μM DHA+0.5 mM VPA and preincubated with HCoV-229E, and (FIG. 6H) MRC5 infected with HCoV-229E (no drug).

FIGS. 7A-B show impact of VPA and DHA on SARS-CoV2 in MRC5 cells. It should be noted however that MRC5 cells lack the receptor for SARS-CoV2, and therefore the amount of replicating virus is very small even without drug. Nonetheless, there is an impact on viral replication with either VPA or DHA and retained in the combination. As the receptor is a clear target for the drug combination, in wildtype infections the impact is expected to be much higher.

FIG. 7A shows the percentage of viral RNA detected by qRT-PCR in MRC5 infected with SARS-CoV2 when treated with VPA, DHA, and VPA+DHA. FIG. 7B shows the percentage of normalized viral RNA detected by RNASeq from the cell lysates treated with VPA, DHA and VPA+DHA. These data clearly demonstrate that the combination of VPA, and DHA on SARS-CoV2 replication is diminished in MRC5 cells, even when the normal receptor for infection is absent.

FIGS. 8A-B show the IC50 and maximum induction of VPA using Vero cells. FIG. 8A shows the IC50 curve of VPA in directly inhibiting HDAC2 activity in Vero cells. FIG. 8B shows the top-Protein expression level of p21 upon treatment of VPA at different time points. Bottom-Time course induction level of p21 by 2.55 mM of VPA in Vero cells.

FIG. 9 shows the distribution of Serum VPA levels during the second quarter of 2021. All VPA levels tested by LabCorp during the second quarter of 2021 were plotted, demonstrating a mean of 56.65 and median of 59, with only 25% of samples testing a value of 79 μg/mL or higher. This indicates that roughly only one-quarter of patients on VPA for standard indications of seizure disorder, bipolar disorder, or migraine headaches were actually in the therapeutic range for their indicated use. Importantly, our data suggest that therapeutic doses for antiviral activity are higher (at least 100 μg/mL) when used alone (without DHA). These data indicate less than one-quarter of the patients on VPA would have had levels sufficient to provide antiviral protection.

FIGS. 10A-B show the characteristics of patients in the Optum dataset listed as having been prescribed VPA in the retrospective cohort of patients tested for COVID-19. FIG. 10A shows the demographics and collection period. FIG. 10B shows the vaccine status and comorbidities.

FIG. 11 shows the odds ratio (OR) of “exposure to VPA prescription” in patients diagnosed with COVID-19 in the retrospective cohort of patients tested for COVID-19.

FIGS. 12A-B show the characteristics of patients prescribed or not prescribed VPA in the retrospective cohort of COVID-19 infected patients. FIG. 12A shows the demographics and collection period. FIG. 12B shows vaccine status and comorbidities.

FIG. 13 shows the clinical progression of COVID-19, in patients prescribed or not prescribed VPA in the COVID-19 infected cohort.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “of” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” can refer to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been identified with having a suspected coronavirus exposure, having a coronavirus infection or being susceptible to a coronavirus infection, such as, for example, prior to the administration step.

As used herein, the term “comprising” can include the aspects “consisting of” and “consisting essentially of.”

A “SARS virus protein” refers to any protein of any SARS virus strain or its functional equivalent as defined herein. Thus, the invention includes, but is not limited to, SARS polymerase, the S (spike) protein, the N (nucleocapsid) protein, the M (membrane) protein, the small envelope E protein, the Nsp (non-structural proteins), and their functional equivalents.

As used herein, the term “contacting” can refer to the placement in direct physical association; includes both in solid and liquid form. “Contacting” is often used interchangeably with “exposed.” In some aspects, “contacting” refers to delivering or exposing a cell to a molecule (such as valproic acid and/or docosahexaenoic acid).

As used herein, the terms “synergy”, “synergism” or “synergistic” mean more than the expected additive effect of a combination. A synergistic effect may be attained when the active ingredients are: (1) co-formulated and administered or delivered simultaneously in a combined, unit dosage formulation; (2) delivered by alternation or in parallel as separate formulations; or (3) by some other regimen.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a coronavirus infection or the disease, disorder, and/or condition can be associated with a coronavirus infection (e.g., COVID-19).

As used herein, the terms “inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels. Further, the terms, “inhibit” or “inhibiting” mean decreasing viral colonization from the amount of colonization that would occur without treatment and/or causing an infection to decrease. Inhibiting also include causing a complete regression of the colonization.

As used herein, “modulate” is meant to mean to alter, by increasing or decreasing.

As used herein, “prevent” is meant to mean minimize the chance that a subject who has an increased susceptibility for developing an infection will develop an infection. In some aspects, the subject has an increased susceptibility for developing an infection because the subject was exposed to one or more viruses disclosed herein. In some aspects, the term “prevent” can also mean minimizing the chance that a subject who has been exposed to one or more viruses disclosed herein will develop a disease, disorder, and/or condition associated with a coronavirus infection (e.g., COVID-19).

As used herein, “treat” is meant to mean administer a compound, composition or molecule of the invention to a subject, such as a human or other mammal (for example, an animal model), that has an infection, in order to prevent or delay a worsening of the effects of the disease or condition, or to partially or fully reverse the effects of the disease.

As used herein, “effective amount” of a compound is meant to mean a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

Valproic acid is a short branched-chain fatty acid, used initially as an excipient for many decades, as it was thought to be inert until it was discovered to have antiseizure activity in the early 1960s (Meunier, H., et al., J. Therapie, 1963. 18: p. 435-8). Subsequently, it was developed as a therapeutic for seizure disorders, and later bipolar disorder (Koch-Weser, J. and T. R. Browne, N Engl J Med, 1980. 302(12): p. 661-6; and Calabrese, J. R. and M. J. Woyshville, J Clin Psychiatry, 1995. 56 Suppl 3: p. 11-8), as well as migraine headaches. While it has a well-established toxicity profile, it has been found to elevate the risk of certain birth defects roughly three-fold and is contraindicated for use in pregnant women (Wartman, C. and A. VandenBerg, Ann Pharmacother, 2022: p. 10600280221085991). The mechanism of action in causing birth defects is not fully understood; however, inhibition of HDAC activity has been proposed (Gottlicher, M., Ann Hematol, 2004. 83 Suppl 1: p. S91-2). This HDAC inhibitory (HDACi) activity has been studied for potential use as cancer therapeutics (Xia, Q., et al., Cancer Res, 2006. 66(14): p. 7237-44; and Shabbeer, S., et al., Prostate, 2007. 67(10): p. 1099-110) and as a treatment strategy for HIV, where it can stimulate the emergence of latent infection from a lysogenous state-theoretically permitting the potential for eradication of disease when used in combination with HIV Highly Active Anti-retroviral Therapy (HAART) (Crosby, B. and C. M. Deas, J Clin Pharm Ther, 2018. 43(5): p. 740-74). Valproic acid has been found to have antiviral activity for various viruses, including DNA and RNA viruses, with enveloped viruses appearing to be the most susceptible (Vazquez-Calvo, A., et al., J Virol, 2011. 85(3): p. 1267-74).

Coronaviruses are a group of RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. In cows and pigs they cause diarrhea, while in mice they cause hepatitis and encephalomyelitis.

Coronaviruses are members of the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

Over the past two decades, emerging pathogenic coronaviruses capable of causing life-threatening disease in humans and animals have been identified, namely severe acute respiratory syndrome coronavirus (SARS-CoV) and Middle Eastern respiratory syndrome coronavirus (MERS-CoV). In December 2019, the Wuhan Municipal Health Committee (Wuhan, China) identified an outbreak of viral pneumonia cases of unknown cause. Coronavirus RNA was identified in some of these patients. The new coronavirus has been named SARS-CoV-2, and the disease caused by this virus has been named COVID-19.

In order to identify coronavirus antiviral therapeutics, many FDA-approved drugs were assessed for potential repurposing against COVID-19. Initial results demonstrated dozens of potential agents predicted to impact coronavirus replication (Gordon, D. E., et al., bioRxiv, 2020; Hsieh, K., et al., Sci Rep, 2021. 11(1): p. 23179; Huang, R., et al., bioRxiv, 2020; and Jeon, S., et al., Antimicrob Agents Chemother, 2020. 64(7)). In vitro testing identified multiple lead agents. Valproic acid (VPA) was identified in this initial screen, as it was a known inhibitor of HDAC2 and shown to interact with nsp5 of SARS-CoV2. Unfortunately, in vitro, viral replication assays failed to demonstrate any antiviral activity of VPA at the doses tested. As described herein in vitro testing of VPA was conducted against coronavirus replication and confirmed those initial findings, and demonstrate antiviral activity at the doses bracketed around the known IC₅₀ for HDAC2 inhibition; however, that dose is too high for clinical use. The results described herein also demonstrate that epidemiologic data support the case for VPA as an antiviral agent against coronavirus despite the predicted lack of activity from high throughput screening assays. The screening methods were investigated, and a surrogate less pathogenic coronavirus (HCoV-229E) was used to dissect potential antiviral pathways, and identify a method to augment valproic acid antiviral activity with the combination of VPA and docosahexaenoic acid (DHA). These findings support the use of VPA/DHA against coronaviruses.

### Methods of Treatment

Disclosed herein are methods of treating a subject having a coronavirus infection (e.g., SARS-CoV-2). In some aspects, the methods can comprise administering to the subject one or more therapeutically effective doses of valproic acid and docosahexaenoic acid. Disclosed herein are methods of treating a subject having a coronavirus infection, the method comprising administering to the subject one or more therapeutically effective doses of valproic acid and docosahexaenoic acid. In some aspects, valproic acid and docosahexaenoic acid can inhibit replication of the coronavirus. Disclosed herein are methods of treating a subject having a coronavirus infection, the methods comprising administering to the subject one or more therapeutically effective doses of (a) depsipeptide and (b) docosahexaenoic acid. Disclosed herein are methods of treating a subject having a coronavirus infection, the methods comprising administering to the subject one or more therapeutically effective doses of depsipeptide and docosahexaenoic acid. Disclosed herein are methods of treating a subject having a coronavirus infection, the methods comprising administering to the subject one or more therapeutically effective doses of trichostatin or a derivative of trichostatin (e.g., suberoylanilide hydroxamic acid) and docosahexaenoic acid. In some aspects, the methods can comprise administering to the subject one or more therapeutically effective doses of (a) depsipeptide and (b) docosahexaenoic acid. In some aspects, (a) depsipeptide and (b) docosahexaenoic acid can inhibit replication of the coronavirus. In some aspects, the methods can further comprise administering to the subject one or more therapeutically effective doses of remdesivir. In some aspects, the subject is infected or has previously been infected with the coronavirus.

Disclosed herein are methods of treating or preventing COVID-19 in a subject. Disclosed herein are methods of treating or preventing COVID-19 in a subject, the methods comprising administering to the subject one or more therapeutically effective doses of valproic acid and docosahexaenoic acid. Also disclosed herein are methods of treating or preventing a disease resulting from a coronavirus infection. In some aspects, the disease resulting from a coronavirus infection is COVID-19. In some aspects, the methods can comprise administering to the subject one or more therapeutically effective doses of valproic acid and docosahexaenoic acid. In some aspects, valproic acid and docosahexaenoic acid can inhibit replication of the coronavirus. Disclosed herein are methods of treating or preventing COVID-19 in a subject, the methods comprising administering to the subject one or more therapeutically effective doses of (a) depsipeptide and (b) docosahexaenoic acid. Disclosed herein are methods of treating or preventing COVID-19 in a subject, the methods comprising administering to the subject one or more therapeutically effective doses of depsipeptide and docosahexaenoic acid. Disclosed herein are methods of treating or preventing COVID-19 in a subject, the methods comprising administering to the subject one or more therapeutically effective doses of trichostatin or a derivative of trichostatin (e.g., suberoylanilide hydroxamic acid) and docosahexaenoic acid. In some aspects, the methods can comprise administering to the subject one or more therapeutically effective doses of (a) depsipeptide and (b) docosahexaenoic acid. In some aspects, (a) depsipeptide and (b) docosahexaenoic acid can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of preventing or inhibiting a coronavirus infection in a subject. In some aspects, the methods can comprise administering to the subject one or more therapeutically effective doses of valproic acid and docosahexaenoic acid. Disclosed herein are methods of preventing or inhibiting a coronavirus infection in a subject, the methods, comprising administering to the subject one or more therapeutically effective doses of valproic acid and docosahexaenoic acid. In some aspects, valproic acid and docosahexaenoic acid can inhibit replication of the coronavirus. Disclosed herein are method of preventing or inhibiting a coronavirus infection in a subject, the methods comprising administering to the subject one or more therapeutically effective doses of (a) depsipeptide and (b) docosahexaenoic acid. Disclosed herein are method of preventing or inhibiting a coronavirus infection in a subject, the methods comprising administering to the subject one or more therapeutically effective doses of depsipeptide and docosahexaenoic acid. Disclosed herein are method of preventing or inhibiting a coronavirus infection in a subject, the methods comprising administering to the subject one or more therapeutically effective doses of trichostatin or a derivative of trichostatin (e.g., suberoylanilide hydroxamic acid) and docosahexaenoic acid. In some aspects, the methods can comprise administering to the subject one or more therapeutically effective doses of (a) depsipeptide and (b) docosahexaenoic acid. In some aspects, (a) depsipeptide and (b) docosahexaenoic acid can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of inhibiting replication of a coronavirus in a subject having a coronavirus infection. In some aspects, the methods can comprise administering to the subject one or more therapeutically effective doses of valproic acid and docosahexaenoic acid. Disclosed herein are methods of inhibiting replication of a coronavirus in a subject having a coronavirus infection, the methods comprising administering to the subject one or more therapeutically effective doses of valproic acid and docosahexaenoic acid. In some aspects, valproic acid and docosahexaenoic acid can inhibit replication of the coronavirus. Disclosed herein are methods of inhibiting replication of a coronavirus in a subject having a coronavirus infection, the methods comprising administering to the subject one or more therapeutically effective doses of (a) depsipeptide and (b) docosahexaenoic acid. Disclosed herein are methods of inhibiting replication of a coronavirus in a subject having a coronavirus infection, the methods comprising administering to the subject one or more therapeutically effective doses of depsipeptide and docosahexaenoic acid. Disclosed herein are methods of inhibiting replication of a coronavirus in a subject having a coronavirus infection, the methods comprising administering to the subject one or more therapeutically effective doses of trichostatin or a derivative of trichostatin (e.g., suberoylanilide hydroxamic acid) and docosahexaenoic acid. In some aspects, the methods can comprise administering to the subject one or more therapeutically effective doses of (a) depsipeptide and docosahexaenoic acid. In some aspects, (a) depsipeptide and docosahexaenoic acid can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of inhibiting replication of a coronavirus in a cell. In some aspects, the methods can comprise contacting the cell infected with the coronavirus with one or more therapeutically effective doses of valproic acid and docosahexaenoic acid. Disclosed herein are methods of inhibiting replication of a coronavirus in a cell, the methods comprising contacting the cell infected with the coronavirus with one or more therapeutically effective doses of valproic acid and docosahexaenoic acid. In some aspects, the methods can comprise delivering to the cell infected with the coronavirus one or more therapeutically effective doses of valproic acid and docosahexaenoic acid. Disclosed herein are method of inhibiting replication of a coronavirus in a cell, the methods comprising contacting the cell infected with the coronavirus with one or more therapeutically effective doses of (a) depsipeptide and (b) docosahexaenoic acid. Disclosed herein are method of inhibiting replication of a coronavirus in a cell, the methods comprising contacting the cell infected with the coronavirus with one or more therapeutically effective doses of depsipeptide and docosahexaenoic acid. Disclosed herein are method of inhibiting replication of a coronavirus in a cell, the methods comprising contacting the cell infected with the coronavirus with one or more therapeutically effective doses of trichostatin or a derivative of trichostatin (e.g., suberoylanilide hydroxamic acid) and docosahexaenoic acid. In some aspects, the methods can comprise contacting the cell infected with the coronavirus with one or more therapeutically effective doses of (a) depsipeptide and (b) docosahexaenoic acid. In some aspects, the methods can comprise delivering to the cell infected with the coronavirus one or more therapeutically effective doses of (a) depsipeptide and (b) docosahexaenoic acid. In some aspects, the methods can further comprise contacting the cell or delivering to the cell infected with the coronavirus one or more therapeutically effective doses of remdesivir.

Disclosed herein are methods of inhibiting, treating or preventing a coronavirus infection in a subject. In some aspects, the methods can comprise administering to the subject having said infection a plurality of therapeutically effective doses of valproic acid and docosahexaenoic acid. In some aspects, valproic acid and docosahexaenoic acid can inhibit replication of the coronavirus. Disclosed herein are methods of method of inhibiting, treating or preventing a coronavirus infection in a subject, the methods comprising administering to the subject having said infection a plurality of therapeutically effective doses of (a) depsipeptide and (b) docosahexaenoic acid. Disclosed herein are methods of method of inhibiting, treating or preventing a coronavirus infection in a subject, the methods comprising administering to the subject having said infection a plurality of therapeutically effective doses of depsipeptide and docosahexaenoic acid. Disclosed herein are methods of method of inhibiting, treating or preventing a coronavirus infection in a subject, the methods comprising administering to the subject having said infection a plurality of therapeutically effective doses of trichostatin or a derivative of trichostatin (e.g., suberoylanilide hydroxamic acid) and docosahexaenoic acid. In some aspects, the methods can comprise administering to the subject having said infection a plurality of therapeutically effective doses of (a) depsipeptide and (b) docosahexaenoic acid. In some aspects, (a) depsipeptide and (b) docosahexaenoic acid can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus. In some aspects, the plurality of therapeutically effective doses of valproic acid and docosahexaenoic acid can be one or more doses administered per day for two or more days per week. In some aspects, the plurality of therapeutically effective doses of valproic acid and docosahexaenoic acid can be one or more doses administered per day for three or more days per week. In some aspects, the plurality of therapeutically effective doses of (a) depsipeptide and (b) docosahexaenoic acid can be one or more doses administered per day for two or more days per week. In some aspects, the plurality of therapeutically effective doses of (a) depsipeptide and (b) docosahexaenoic acid can be one or more doses administered per day for three or more days per week. In some aspects, the dosing can be continued for one or more weeks per month. In some aspects, the dosing can be continued for one or more months per year.

Disclosed herein are methods of reducing one or more symptoms of a coronavirus infection or COVID-19 in a subject. In some aspects, the methods can comprise administering to the subject one or more therapeutically effective doses of valproic acid and docosahexaenoic acid. Disclosed herein are methods of reducing one or more symptoms of a coronavirus infection or COVID-19 in a subject, the methods comprising administering to the subject one or more therapeutically effective doses of valproic acid and docosahexaenoic acid. Disclosed herein are methods of reducing one or more symptoms of a coronavirus infection or COVID-19 in a subject, the methods comprising administering to the subject one or more therapeutically effective doses of (a) depsipeptide and (b) docosahexaenoic acid. Disclosed herein are methods of reducing one or more symptoms of a coronavirus infection or COVID-19 in a subject, the methods comprising administering to the subject one or more therapeutically effective doses of depsipeptide and docosahexaenoic acid. Disclosed herein are methods of reducing one or more symptoms of a coronavirus infection or COVID-19 in a subject, the methods comprising administering to the subject one or more therapeutically effective doses of trichostatin or a derivative of trichostatin (e.g., suberoylanilide hydroxamic acid) and docosahexaenoic acid. In some aspects, the methods can comprise administering to the subject one or more therapeutically effective doses of (a) depsipeptide and (b) docosahexaenoic acid. In some aspects, the one or more symptoms the coronavirus infection or COVID-19 are fever, sore throat, malaise, difficulty breathing, fatigue, muscle aches, or difficulty with cognition or concentration. In some aspects, valproic acid and docosahexaenoic acid can inhibit replication of the coronavirus. In some aspects, (a) depsipeptide and (b) docosahexaenoic acid can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus. In some aspects, the plurality of therapeutically effective doses of valproic acid and docosahexaenoic acid can be one or more doses administered per day for two or more days per week. In some aspects, the plurality of therapeutically effective doses of valproic acid and docosahexaenoic acid can be one or more doses administered per day for three or more days per week. In some aspects, the plurality of therapeutically effective doses of (a) depsipeptide and (b) docosahexaenoic acid can be one or more doses administered per day for two or more days per week. In some aspects, the plurality of therapeutically effective doses of (a) depsipeptide and (b) docosahexaenoic acid can be one or more doses administered per day for three or more days per week. In some aspects, the dosing can be continued for one or more weeks per month. In some aspects, the dosing can be continued for one or more months per year.

In some aspects, valproic acid and docosahexaenoic acid can be administered to the subject immediately after infection or any time within one day to 5 days after infection, at the earliest time after diagnosis of infection with the coronavirus, or at the time of exposure to a coronavirus (e.g., before the appearance of one or more symptoms of infection).

In some aspects, (a) depsipeptide and (b) docosahexaenoic acid can be administered to the subject immediately after infection or any time within one day to 5 days after infection, at the earliest time after diagnosis of infection with the coronavirus, or at the time of exposure to a coronavirus (e.g., before the appearance of one or more symptoms of infection).

In some aspects, valproic acid and docosahexaenoic acid can be administered to the subject as a primary antiviral therapy, adjunct antiviral therapy, or a co-antiviral therapy, or wherein the administration comprises separate administration or coadministration of valproic acid and docosahexaenoic acid with at least one other antiviral composition or with at least one other composition for treating one or more symptoms associated with said coronavirus infection. In some aspects, the at least one other antiviral composition can be remdesivir.

In some aspects, (a) depsipeptide and (b) docosahexaenoic acid can be administered to the subject as a primary antiviral therapy, adjunct antiviral therapy, or a co-antiviral therapy, or wherein the administration comprises separate administration or coadministration of (a) depsipeptide and (b) docosahexaenoic acid with at least one other antiviral composition or with at least one other composition for treating one or more symptoms associated with said coronavirus infection. In some aspects, the at least one other antiviral composition can be remdesivir.

In some aspects, the subject can be a human subject. In some aspects, the cell can be a mammalian cell. In some aspects, the mammalian cell can be a human cell. In some aspects, the subject is not infected and has not previously been infected with the coronavirus. In some aspects, the subject is at risk of being infected with the coronavirus. In some aspects, the subject has been exposed to or is suspected of being exposed to a coronavirus. In some aspects, the subject has not been exposed to or is not suspected of being exposed to a coronavirus. In some aspects, the subject has an active infection. In some aspects, the subject has had a prior infection. In some aspects, the subject does not have a seizure disorder. In some aspects, the subject does not have a bipolar disorder. In some aspects, the subject does not have a seizure disorder or bipolar disorder.

Any of the methods described herein can include the step of determining whether or not the subject has a viral infection; indicating administration of valproic acid (or depsipeptide) and docosahexaenoic acid; administering an initial dose of valproic acid (or depsipeptide) and docosahexaenoic acid to the subject according to a prescribed initial dosing regimen for a period of time; periodically determining the adequacy of subject's clinical response and/or therapeutic response to treatment with valproic acid (or depsipeptide) and docosahexaenoic acid; and if the subject's clinical response and/or therapeutic response is adequate, then continuing treatment with valproic acid (or depsipeptide) and docosahexaenoic acid as needed until the desired clinical endpoint is achieved; or if the subject's clinical response and/or therapeutic response are inadequate at the initial dose and initial dosing regimen, then escalating or deescalating the dose until the desired clinical response and/or therapeutic response in the subject is achieved.

Treatment of the subject with valproic acid (or depsipeptide) and docosahexaenoic acid can be continued as needed. The dose or dosing regimen can be adjusted as needed until the patient reaches the desired clinical endpoint(s) such as a reduction or alleviation of specific symptoms associated with the viral infection. Determination of the adequacy of clinical response and/or therapeutic response can be conducted by a clinician familiar with viral infections.

In some aspects, the coronavirus can be pathogenic to humans. In some aspects, the coronavirus can be severe acute respiratory syndrome coronavirus 2 (SARS-CoV-2), severe acute respiratory syndrome (SARS), middle east respiratory syndrome (MERS), human coronavirus 229E, human coronavirus NL63, Miniopterus bat coronavirus 1, Miniopterus bat coronavirus HKU8, porcine epidemic diarrhea virus, Rhinolophus bat coronavirus HKU2, Scotophilus bat coronavirus 512, bovine coronavirus, human coronavirus OC43, human coronavirus HKU1, murine coronavirus, Pipistrellus bat coronavirus HKU5, Rousettus bat coronavirus HKU9, Tylonycteris bat coronavirus HKU4, hedgehog coronavirus 1, infectious bronchitis virus, beluga whale coronavirus SW1, infectious bronchitis virus, Bulbul coronavirus HKU11, pangolin coronavirus, porcine coronavirus HKU15, WIV1-CoV, SHC014-CoV, bat-SL-CoVZC45, bat-SLCoVZXC21, SARS-CoVGZ02, BtKY72, WIV16, Rs4231, Rs7327, Rs9401, BtRs-BetaCoV/YN2018R, BtRs-BetaCoV/YN2013, Anlong-112, Rf2092, BtRs-BetaCoV/YN2018C, As6526, Rs4247, BtRs-BetaCoV/GX2013, Yunnan2011, BtRl-BetaCoV/SC2018, Shannxi2011, BtRs-BetaCoV/HuB2013, Bat_CoV_279/2005, HKU3-12, HKU3-3, HKU3-7, Longquan-140, or RaTG13.

In some aspects, the coronavirus can be wild type SARS-CoV-2 or a variant strain thereof such as, but not limited to, the variants of D614G (originally found in China/Germany), B.1.1.7 or 20I/501Y.V1 (originally found in the United Kingdom), B.1.351 or 20H/501.V2 (originally found in South Africa), P.1 or 20J/501Y.V3 (originally found in Japan/Brazil), 20C/S:452R (originally found in California), Cluster 5 Variant (originally found in Denmark), XBB.1.5 (e.g., 1.5.70, 1.5.68, 1.5.72, 1.5.10, 1.5.59, 1.5.1), XBB.1.16 (e.g., 1.16.6, 1.16.11, 1.16.15, 1.6.1), EG.5, XBB.1.9 (e.g., 1.9.1, 1.9.2), XBB.2.3, BA.1, BA.5, BA.2.86, HV.1, FL.1.5.1, HK.3, JD.1.1, JF.1, GK.1.1., HF.1, BA.2 (e.g., 2.12.1, 2.12.2), BA.4, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1 (e.g., 1.1). In some aspects, a SARS-CoV-2 S protein can be derived from any of the SARS-CoV-2 strains of a, 3, 7, 6, or F (original or subvariant strains).

In some aspects, the coronavirus can be SARS-CoV, MERS-CoV, SARS-CoV-2, HCoV 229E, HCoV NL63, HCoV OC43, or HCoV HKU1.

In some aspects, the coronavirus can be SARS-CoV, MERS-CoV, SARS-CoV-2, HCoV 229E, HCoV NL63, HCoV OC43, HCoV HKU1, D614G, B.1.1.7, 20I/501Y.V1, B.1.351 or 20H/501.V2, P.1, 20J/501Y.V3, 20C/S:452R, Cluster 5 Variant, XBB.1.5, XBB.1.5.70, XBB.1.5.68, XBB.1.5.72, XBB.1.5.10, XBB.1.5.59, XBB.1.5.1, XBB.1.16, XBB.1.16.6, XBB.1.16.11, XBB.1.16.15, XBB.1.6.1, EG.5, XBB.1.9, XBB.1.9.1, XBB.1.9.2, XBB.2.3, BA.1, BA.5, BA.2.86, HV.1, FL.1.5.1, HK.3, JD.1.1, JF.1, GK.1.1., HF.1, BA.2, BA.2.12.1, BA.2.12.2, BA.4, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1, or BQ.1.1.

In some aspects, the coronavirus can be any member of the alpha and beta coronaviruses capable of causing human infection.

### Compositions

The compositions described herein can be formulated to include a therapeutically effective dose (or amount) of valproic acid (or depsipeptide) and docosahexaenoic acid. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of coronavirus.

The compositions described herein can be formulated in a variety of combinations. The particular combination of valproic acid (or depsipeptide) and docosahexaenoic acid with one or more antivirals can vary according to many factors, for example, the particular the type and severity of the infection. In some aspects, the antiviral can be remdesivir.

The compositions described herein can be administered to the subject (e.g., a human patient) in an amount sufficient to delay, reduce, or preferably prevent the onset of infection or clinical disease. Accordingly, in some aspects, the patient is a human patient. In therapeutic applications, compositions can be administered to a subject (e.g., a human patient) already with or diagnosed with an infection or a disease associated with the infection in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the infection (e.g., replication of the coronavirus) is delayed, hindered, or prevented, or the infection or a symptom of the infection or disease associated with the infection is ameliorated. One or more of the symptoms can be less severe. Recovery can be accelerated in an individual who has been treated.

Disclosed herein, are methods of treating a subject with a coronavirus infection. The coronavirus can be any coronavirus. In some aspects, the coronavirus is SARS-CoV, MERS-CoV, SARS-CoV-2, HCoV 229E, HCoV NL63, HCoV OC43, HCoV HKU1. In some aspects, the coronavirus is SARS-CoV, MERS-CoV, SARS-CoV-2, HCoV 229E, HCoV NL63, HCoV OC43, HCoV HKU1, D614G, B.1.1.7, 20I/501Y.V1, B.1.351 or 20H/501.V2, P.1, 20J/501Y.V3, 20C/S:452R, Cluster 5 Variant, XBB.1.5, XBB.1.5.70, XBB.1.5.68, XBB.1.5.72, XBB.1.5.10, XBB.1.5.59, XBB.1.5.1, XBB.1.16, XBB.1.16.6, XBB.1.16.11, XBB.1.16.15, XBB.1.6.1, EG.5, XBB.1.9, XBB.1.9.1, XBB.1.9.2, XBB.2.3, BA.1, BA.5, BA.2.86, HV.1, FL.1.5.1, HK.3, JD.1.1, JF.1, GK.1.1., HF.1, BA.2, BA.2.12.1, BA.2.12.2, BA.4, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1, or BQ.1.1.

In some aspects, the subject has been diagnosed with a coronavirus infection or a disease associated with a coronavirus infection (e.g., COVID-19) prior to the administering step.

The compositions described herein can be formulated to include a therapeutically effective amount of valproic acid (or depsipeptide) and docosahexaenoic acid alone or in combination with one or more antivirals (e.g., remdesivir). In some aspects, valproic acid (or depsipeptide) and docosahexaenoic acid can be contained within a pharmaceutical formulation. In some aspects, valproic acid (or depsipeptide) and docosahexaenoic acid can be contained separately within a pharmaceutical formulation. In some aspects, the pharmaceutical formulation can be a unit dosage formulation. In some aspects, the antiviral therapeutic can be a cellular or gene therapy therapeutic, an immunomodulatory, an antibody or mixture of antibodies or an antiviral. In some aspects, the antiviral therapeutic is remdesivir (Veklury), Nafamostat, Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib).

In some aspects, the antiviral therapeutic can be a quinoline-based antimalarial ((hydroxy)-chloroquine and others), RAAS modifiers (captopril, losartan, and others), statins (atorvastatin and simvastatin), guanidino-based serine protease inhibitors (camostat and nafamostat), antibacterials (macrolides, clindamycin, and doxycycline), antiparasitics (ivermectin and niclosamide), cardiovascular drugs (amiodarone, verapamil, and tranexamic acid), antipsychotics (chlorpromazine), antivirals (umifenovir and oseltamivir), DPP-4 inhibitors (linagliptin), JAK inhibitors (baricitinib and others), sulfated glycosaminoglycans (UFH and LMWHs) and polypeptides such as the enzymes DAS181 and rhACE2. They also include the viral spike protein-targeting monoclonal antibodies REGN10933 and REGN10987.

The therapeutically effective amount or dosage of valproic acid (or depsipeptide) and docosahexaenoic acid alone or in combination with remdesivir used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms of the infection), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the valproic acid (or depsipeptide) and docosahexaenoic acid can be administered daily (including multiple times in the same day); once a week (for, for example, 2 or more weeks to many months or years); once a month (for, for example, two to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly. In some aspects, for example, the remdesivir can be administered to subjects with COVID-19 intravenously, 200 mg, as a single dose on day 1, followed by 100 mg once daily. It is also noted that the frequency of treatment can be variable, and depend on several factors including but not limited to oxygen requirements and need for ventilatory support. For example, the present compositions can be administered once (or twice, three times, etc.) daily, weekly, monthly, or yearly.

In some aspects, valproic acid (or depsipeptide) and docosahexaenoic acid can be administered to the subject immediately after infection or any time within one day to 5 days after infection, at the earliest time after diagnosis of infection with the coronavirus, or after a potential exposure to the coronavirus.

In some aspects, valproic acid (or depsipeptide) and docosahexaenoic acid can be administered to the subject as a primary antiviral therapy, adjunct antiviral therapy, or a co-antiviral therapy, or wherein the administration comprises separate administration or coadministration of valproic acid and docosahexaenoic acid with at least one other antiviral composition or with at least one other composition for treating one or more symptoms associated with said coronavirus infection. In some aspects, the at least one other antiviral composition can be remdesivir.

Dosages of valproic acid can be in the range of 500 mg to 2,500 mg/day. In some aspects, the dosage of valproic acid can be 500, 525, 550, 575, 600, 625, 650, 675, 700, 725, 750, 775, 800, 825, 850, 875, 900, 925, 950, 975, 1,000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500 mg total or any amount in between. In some aspects, the therapeutically effective dose of valproic acid may be less when combined with docosahexaenoic acid or combined with docosahexaenoic acid and remdesivir. In some aspects, the administration of valproic acid increases the efficacy of docosahexaenoic acid and/or remdesivir. In some aspects, the valproic acid total dose per day can be independently selected upon each occurrence from about 500 mg to about 2000 mg.

Dosages of depsipeptide can be in the range of 1 mg/m² to 15 mg/m². In some aspects, the dosage of depsipeptide acid can be 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 mg/m² total or any amount in between. In some aspects, the therapeutically effective dose of depsipeptide may be less when combined with docosahexaenoic acid or combined with docosahexaenoic acid and remdesivir. In some aspects, the administration of depsipeptide increases the efficacy of docosahexaenoic acid and/or remdesivir. In some aspects, the depsipeptide total dose per day can be independently selected upon each occurrence from about 1 mg/m² to 10 mg/m². In some aspects, the depsipeptide total dose per day can be about 13 mg/m².

In some aspects, the derivative of trichostatin can be suberoylanilide hydroxamic acid (SAHA, also referred to as Vorinostat or Zolinza). Dosages of SAHA can be in the range of 100 mg to 600 mg/day. In some aspects, the dosage of SAHA can be 100, 200, 300, 400, 500, 600 mg total or any amount in between. In some aspects, the therapeutically effective dose of SAHA may be less when combined with docosahexaenoic acid or combined with docosahexaenoic acid and remdesivir. In some aspects, the administration of SAHA increases the efficacy of docosahexaenoic acid and/or remdesivir. In some aspects, the SAHA total dose per day can be independently selected upon each occurrence from about 100 mg to about 600 mg. In some aspects, the dose of SAHA can be 200 mg twice a day or 300 mg twice a day.

Dosages of docosahexaenoic acid can be in the range of 500 mg to 1,000 mg/day. In some aspects, the dosage of docosahexaenoic acid can be 500, 525, 550, 575, 600, 625, 650, 675, 700, 725, 750, 775, 800, 825, 850, 875, 900, 925, 950, 975, or 1,000, mg total or any amount in between. In some aspects, the therapeutically effective dose of docosahexaenoic acid may be less when combined with valproic acid (or depsipeptide) or combined with valproic acid (or depsipeptide) and remdesivir. In some aspects, the administration of docosahexaenoic acid increases the efficacy of valproic acid (or depsipeptide) and/or remdesivir. In some aspects, the docosahexaenoic acid total dose per day can be independently selected upon each occurrence from about 500 mg to about 1000 mg.

Dosages of remdesivir can be in the range of 100 to 200 mg total or any amount in between. In some aspects, 200 mg of remdesivir can be administered as a single dose. In some aspects, 100 mg of remdesivir can be administered as a single dose. Suitable treatment regimens using any of the dosages described herein include but are not limited to 200 mg of remdesivir can be administered as a single dose on day 1, followed by 100 mg once daily for 2 or more days. In some aspects, 200 mg of remdesivir can be administered as a single dose on day 1, followed by 100 mg once daily for 2 or more days. In some aspects, 200 mg of remdesivir can be administered as a single dose on day 1, followed by 100 mg once daily for 3 or more days. In some aspects, 200 mg of remdesivir can be administered as a single dose on day 1, followed by 100 mg once daily for 4 days. In some aspects, 200 mg of remdesivir can be administered as a single dose on day 1, followed by 100 mg once daily for up to 4 days (or more) or until the subject is discharged from the hospital. In some aspects, In some aspects, 200 mg of remdesivir can be administered as a single dose on day 1, followed by 100 mg once daily up to 5, 6, 7, 8, 9 or 10 days. In some aspects, 200 mg of remdesivir can be administered as a single dose on day 1, followed by 100 mg once daily up to 10 days in patients without substantial clinical improvement at day 5 (Beigel 2020; FDA 2020a; NIH 2020). In some aspects, remdesivir can be administered in combination with prednisone. In some aspects, the therapeutically effective dose of remdesivir may be less when combined with valproic acid (or depsipeptide) and docosahexaenoic acid.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure. In some aspects, the valproic acid can be administered by a different route than docosahexaenoic acid. In some aspects, valproic acid and docosahexaenoic acid can be co-formulated. In some aspects, the depsipeptide can be administered by a different route than docosahexaenoic acid. In some aspects, (a) depsipeptide or (b) trichostatin or a derivative of trichostatin (e.g., suberoylanilide hydroxamic acid) and docosahexaenoic acid can be co-formulated.

In some aspects, the therapeutically effective dose of valproic acid and docosahexaenoic acid can be in a ratio of 2.5:1 to 5:1. In some aspects, the therapeutically effective doses of valproic acid and docosahexaenoic acid can be administered in synergistic combination.

In some aspects, the therapeutically effective dose of depsipeptide acid can be about 1 mg/mg² to about 10 mg/m² and the therapeutically effective dose of docosahexaenoic acid can be about 25 μM or 500 mg to 1,000 mg/day. In some aspects, the therapeutically effective doses of depsipeptide and docosahexaenoic acid can be administered in synergistic combination.

In some aspects, the therapeutically effective dose of suberoylanilide hydroxamic acid can be about 100 mg to 600 mg/day and the therapeutically effective dose of docosahexaenoic acid can be about 25 μM or 500 mg to 1,000 mg/day. In some aspects, the therapeutically effective doses of suberoylanilide hydroxamic acid and docosahexaenoic acid can be administered in synergistic combination.

The compositions described herein can be administered in conjunction with other therapeutic modalities to a subject in need of therapy. The present compounds can be given to prior to, simultaneously with or after treatment with other agents or regimes. For example, valproic acid (or depsipeptide) and docosahexaenoic acid alone or with any of the antivirals disclosed herein can be administered in conjunction with standard therapies used to treat a coronavirus. In some aspects, any of the compounds or compositions described herein can be administered or used together with an anti-inflammatory agent. In some aspects, any of the compounds or compositions described herein can be administered or used together with an immunomodulatory agent. In some aspects, the immunomodulatory agent is not a primary anti-inflammatory agent, and includes but is not limited to statins, estrogen therapy, and antibody therapies.

Any of the compounds or compositions described herein can be administered as a term “combination.” It is to be understood that, for example, valproic acid can be provided to the subject in need, either prior to administration of docosahexaenoic acid, concomitant with administration of docosahexaenoic acid or any combination thereof (co-administration) or shortly thereafter. In some aspects, depsipeptide can be provided to the subject in need, either prior to administration of docosahexaenoic acid, concomitant with administration of docosahexaenoic acid or any combination thereof (co-administration) or shortly thereafter.

In some aspects, valproic acid and docosahexaenoic acid has an additive or synergistic effect with one or more drug classes or antivirals.

In some aspects, (a) depsipeptide and (b) docosahexaenoic acid has an additive or synergistic effect with one or more drug classes or antivirals.

In some aspects, in any of the methods disclosed herein, valproic acid (or depsipeptide) and docosahexaenoic acid can be administered to a subject with one or more therapeutically effective doses of a polymerase inhibitor. In some aspects, the polymerase inhibitor can be molnupiravir, 4′-fluorouridine, favipiravir, or remdesivir. In some aspects, the therapeutically effective doses of valproic acid (or depsipeptide) and docosahexaenoic acid and the polymerase inhibitor can be administered in an additive or synergistic combination.

In some aspects, in any of the methods disclosed herein, valproic acid (or depsipeptide) and docosahexaenoic acid can be administered to a subject with one or more therapeutically effective doses of a protease inhibitor. In some aspects, the protease inhibitor can be nirmatrelvir, ritonavir, a combination of nirmatrelvir and ritonavir, or GC-376. In some aspects, the therapeutically effective doses of valproic acid (or depsipeptide) and docosahexaenoic acid and the protease inhibitor can be administered in an additive or synergistic combination.

In some aspects, in any of the methods disclosed herein, valproic acid (or depsipeptide) and docosahexaenoic acid can be administered to a subject with one or more therapeutically effective doses of a helicase inhibitor. In some aspects, the helicase inhibitor can be cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, or differin. In some aspects, the therapeutically effective doses of valproic acid (or depsipeptide) and docosahexaenoic acid and the helicase inhibitor can be administered in an additive or synergistic combination.

In some aspects, in any of the methods disclosed herein, valproic acid (or depsipeptide) and docosahexaenoic acid can be administered to a subject with one or more therapeutically effective doses of an inhibitor of host proteins supporting viral replication. In some aspects, the inhibitor of host proteins supporting viral replication can be plitidepsin. In some aspects, the therapeutically effective doses of valproic acid (or depsipeptide) and docosahexaenoic acid and the inhibitor of host proteins supporting viral replication can be administered in an additive or synergistic combination.

In some aspects, in any of the methods disclosed herein, valproic acid (or depsipeptide) and docosahexaenoic acid can be administered to a subject with one or more therapeutically effective doses of a non-vaccine biologic. In some aspects, the non-vaccine biologic can be convalescent plasma, actemra, a monoclonal antibody specific for a viral protein. In some aspects, the therapeutically effective doses of valproic acid (or depsipeptide) and docosahexaenoic acid and the non-vaccine biologic can be administered in an additive or synergistic combination.

In some aspects, in any of the methods disclosed herein, valproic acid (or depsipeptide) and docosahexaenoic acid can be administered to a subject with one or more therapeutically effective doses of an inhibitor of viral attachment and entry. In some aspects, the inhibitor of viral attachment and entry can be human recombinant soluble angiotensin converting enzyme-2 (ACE2) or camostate mesylate and analogs thereof. In some aspects, the therapeutically effective doses of valproic acid (or depsipeptide) and docosahexaenoic acid and the inhibitor of viral attachment and entry can be administered in an additive or synergistic combination.

In some aspects, in any of the methods disclosed herein, valproic acid (or depsipeptide) and docosahexaenoic acid can be administered to a subject with one or more therapeutically effective doses of a selective serotonin reuptake inhibitor. In some aspects, the selective serotonin reuptake inhibitor can be fluvoxamine. In some aspects, the therapeutically effective doses of valproic acid (or depsipeptide) and docosahexaenoic acid and the selective serotonin reuptake inhibitor can be administered in an additive or synergistic combination.

In some aspects, in any of the methods disclosed herein, valproic acid (or depsipeptide) and docosahexaenoic acid can be administered to a subject with one or more therapeutically effective doses of one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors. In some aspects, the therapeutically effective doses of valproic acid (or depsipeptide) and docosahexaenoic acid and the one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors can be administered in an additive or synergistic combination.

### Pharmaceutical Compositions

Disclosed herein, are pharmaceutical compositions, comprising one or more of the therapeutic compositions disclosed herein. Disclosed herein, are pharmaceutical compositions, comprising valproic acid and docosahexaenoic acid (e.g., a therapeutically effective dose) and a pharmaceutical acceptable carrier, diluent or excipient as described herein. Disclosed herein, are pharmaceutical compositions, comprising (a) depsipeptide and (b) docosahexaenoic acid (e.g., a therapeutically effective dose) and a pharmaceutical acceptable carrier, diluent or excipient as described herein. Disclosed herein, are pharmaceutical compositions, comprising valproic acid (or depsipeptide) and docosahexaenoic acid (e.g., a therapeutically effective dose) and a pharmaceutical acceptable carrier, diluent or excipient as described herein and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors. In some aspects, valproic acid (or depsipeptide) and docosahexaenoic acid can be formulated for oral or parenteral administration. In some aspects, the parenteral administration is intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes valproic acid (or depsipeptide) or docosahexaenoic acid dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, a pharmaceutical composition comprises valproic acid or docosahexaenoic acid. In some aspects, a pharmaceutical composition comprises valproic acid or docosahexaenoic acid and optionally, a pharmaceutical acceptable carrier, diluent or excipient. In some aspects, a pharmaceutical composition comprises valproic acid and docosahexaenoic acid, and optionally, a pharmaceutical acceptable carrier, diluent or excipient. Disclosed herein, are pharmaceutical compositions, comprising valproic acid and docosahexaenoic acid (e.g., a therapeutically effective dose) and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient.

In some aspects, a pharmaceutical composition comprises depsipeptide or docosahexaenoic acid. In some aspects, a pharmaceutical composition comprises depsipeptide or docosahexaenoic acid and optionally, a pharmaceutical acceptable carrier, diluent or excipient. In some aspects, a pharmaceutical composition comprises depsipeptide and docosahexaenoic acid, and optionally, a pharmaceutical acceptable carrier, diluent or excipient. Disclosed herein, are pharmaceutical compositions, comprising depsipeptide and docosahexaenoic acid (e.g., a therapeutically effective dose) and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient.

In some aspects, a pharmaceutical composition comprises trichostatin or docosahexaenoic acid. In some aspects, a pharmaceutical composition comprises trichostatin or docosahexaenoic acid and optionally, a pharmaceutical acceptable carrier, diluent or excipient. In some aspects, a pharmaceutical composition comprises trichostatin and docosahexaenoic acid, and optionally, a pharmaceutical acceptable carrier, diluent or excipient. In some aspects, a pharmaceutical composition comprises a derivative of trichostatin (e.g., suberoylanilide hydroxamic acid) or docosahexaenoic acid. In some aspects, a pharmaceutical composition comprises a derivative of trichostatin (e.g., suberoylanilide hydroxamic acid) or docosahexaenoic acid and optionally, a pharmaceutical acceptable carrier, diluent or excipient. In some aspects, a pharmaceutical composition comprises a derivative of trichostatin (e.g., suberoylanilide hydroxamic acid) and docosahexaenoic acid, and optionally, a pharmaceutical acceptable carrier, diluent or excipient. Disclosed herein, are pharmaceutical compositions, comprising trichostatin or a derivative of trichostatin (e.g., suberoylanilide hydroxamic acid) and docosahexaenoic acid (e.g., a therapeutically effective dose) and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient.

In some aspects, the pharmaceutical composition can be formulated for oral or intravenous administration. In some aspects, the compositions described herein can be formulated for buccal, enteral, intramuscular, subdermal, sublingual, peroral, oral administration, or a combination thereof.

### Articles of Manufacture

The compositions described herein can be packaged in a suitable container labeled, for example, for use as a therapy to treat, inhibit or prevent a coronavirus infection, inhibit replication of a coronavirus or any of the methods disclosed herein.

Disclosed herein are kits comprising one or more therapeutically effective doses of valproic acid and docosahexaenoic acid for inhibiting, treating or preventing a coronavirus infection in a subject. Disclosed herein are kits comprising one or more therapeutically effective doses of valproic acid and docosahexaenoic acid and one or more therapeutically effective doses of at least one other antiviral for inhibiting, treating or preventing a coronavirus infection in a subject.

Disclosed herein are kits comprising one or more therapeutically effective doses of (a) depsipeptide and (b) docosahexaenoic acid for inhibiting, treating or preventing a coronavirus infection in a subject. Disclosed herein are kits comprising one or more therapeutically effective doses of (a) depsipeptide and (b) docosahexaenoic acid and one or more therapeutically effective doses of at least one other antiviral for inhibiting, treating or preventing a coronavirus infection in a subject.

Disclosed herein are kits for use in treating a subject suffering from a coronavirus infection. In some aspects, the kit comprises: valproic acid and docosahexaenoic acid; and at least one other antiviral. In some aspects, the kit comprises: (a) depsipeptide and (b) docosahexaenoic acid; and at least one other antiviral. In some aspects, the antiviral can be remdesivir (Veklury), Nafamostat. Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, Calquence (acalabrutinib), a quinoline-based antimalarial ((hydroxy)-chloroquine and others), RAAS modifiers (captopril, losartan, and others), statins (atorvastatin and simvastatin), guanidino-based serine protease inhibitors (camostat and nafamostat), antibacterials (macrolides, clindamycin, and doxycycline), antiparasitics (ivermectin and niclosamide), cardiovascular drugs (amiodarone, verapamil, and tranexamic acid), antipsychotics (chlorpromazine), antivirals (umifenovir and oseltamivir), DPP-4 inhibitors (linagliptin), JAK inhibitors (baricitinib and others), sulfated glycosaminoglycans (UFH and LMWHs) and polypeptides such as the enzymes DAS181 and rhACE2, or viral spike protein-targeting monoclonal antibodies REGN10933 and REGN10987. Disclosed herein are kits for use in preventing or inhibiting a coronavirus infection in a subject. Disclosed herein are kits for use in inhibiting replication of a coronavirus infection in a subject. In some aspects, the kits comprise: valproic acid and docosahexaenoic acid; and remdesivir. In some aspects, the kits comprise: (a) depsipeptide and (b) docosahexaenoic acid; and remdesivir. In some aspects, the kits can further comprise at least one pharmaceutically acceptable carrier, diluent or excipient. In some aspects, the kits can further comprise at least one polymerase inhibitor, a protease inhibitor, helicase inhibitor, inhibitor of host proteins supporting viral replication, non-vaccine biologic, inhibitor of viral attachment and entry, or selective serotonin reuptake inhibitor and optionally, a pharmaceutical acceptable carrier, diluent or excipient. In some aspects, the kits can comprise: (a) valproic acid; and (b) docosahexaenoic acid. In some aspects, the kits can comprise: (a) valproic acid; (b) docosahexaenoic acid; (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone. In some aspects, the kits can comprise: (a) valproic acid; (b) docosahexaenoic acid; (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987. In some aspects, the kits can comprise: (a) depsipeptide; and (b) docosahexaenoic acid. In some aspects, the kits can comprise: (a) depsipeptide; (b) docosahexaenoic acid; (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone. In some aspects, the kits can comprise: (a) depsipeptide; (b) docosahexaenoic acid; (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

In some aspects, the kits can further comprise instructions for using valproic acid and docosahexaenoic acid in treating a coronavirus infection. In some aspects, the kits can further comprise instructions for using (a) depsipeptide and (b) docosahexaenoic acid in treating a coronavirus infection. Accordingly, packaged products (e.g., sterile containers containing the composition described herein and packaged for storage, shipment, or sale at concentrated or ready-to-use concentrations) and kits, including at least valproic acid (or depsipeptide) and docosahexaenoic acid as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution. In some aspects, valproic acid and docosahexaenoic acid can be co-packaged. In some aspects, valproic acid and docosahexaenoic acid and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient can be co-packaged. In some aspects, (a) depsipeptide and (b) docosahexaenoic acid can be co-packaged. In some aspects, (a) depsipeptide and (b) docosahexaenoic acid and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient can be co-packaged.

### EXAMPLES

### Example 1: Valproic Acid Use is Associated with Diminished Risk of Contracting COVID-19, and Diminished Disease Severity: Epidemiologic and In Vitro Analysis Reveal Mechanistic Insights

Disclosed herein is the finding that the small branched-chain fatty acid valproic acid (VPA), approved for use in patients with seizure and bipolar disorder, has an anti-coronavirus activity that can be augmented with the addition of a long-chain polyunsaturated omega-3 fatty acid. An epidemiological survey of patients tested for COVID-19 demonstrates a correlation between a reduced infection rate in patients known to be treated with valproic acid of up to 25%, as well as a decreased risk of emergency room visits, hospitalization, ICU admission, and use of mechanical ventilation. In vitro studies demonstrate that valproic acid alters gene expression changes in MRC5 cells, correlating to the inhibition of several SARS-CoV2 interacting genes. Adding the omega-3 fatty acid docosahexaenoic acid (DHA) results in augmentation of viral inhibition for the alpha-coronavirus HCoV-229E (often associated with the “common cold”) and SARS-CoV2. The therapeutic effects of the antiviral combination results from the activation of pre-existing intracellular antiviral mechanisms normally repressed by coronaviruses and can potentially protect against an even broader range of similar viruses. Gene expression profiles demonstrate subtle differences in overall gene expression between VPA-treated and VPA+DHA-treated cells; however, upon infection with HCoV-229E—there is an intensely different response to the infection, as manifested by marked induction of multiple intracellular inflammatory genes. These gene expression changes take 24 or more hours to manifest, which may partly explain why prior drug screens have failed to identify any antiviral activity of VPA, despite in silico predictions. In addition, many of the conventional high throughput cell lines used for SARS-CoV2 screening were previously optimized for viral propogation (Smith, C. D., et al., J Clin Microbiol, 1986. 24(2): p. 265-8) and, in at least one case, appear to lack expression of many of these host response genes (Konishi, K., et al., Front Genet, 2022. 13: p. 801382). The findings disclosed herein demonstrate an interaction between HDAC inhibition and potent activation of cellular antiviral responses and, in combination with a long-chain omega-3 fatty acid DHA, provide the foundation for a low-cost, highly effective antiviral strategy.

Materials and Methods. Cell Culture. MRC-5 cells (ATCC CCL-171) were grown in Eagle's Minimum Essential Medium (ATCC), and Vero cells (ATCC CCL-81) were grown in Dulbecco's Modified Eagle's Medium (ATCC), both with 10% Fetal Bovine Serum (Gibco) and 1% PEN-STREP (Hyclone) at 37° C. in a humidified chamber with 5% CO₂. Cell growth and cytotoxicity of valproic acid (MP Biomedical LLC) were measured using an XTT Assay (ATCC) with standard protocols.

Viral Assays. The IC₅₀ of VPA was determined in Vero and MRC-5 cells using three different assays. For the luciferase assay, Vero cells were seeded at 0.3×10⁶ cells/well in 12-well plates, incubated at 37° C. with 5% CO₂, and grown to ˜90% confluence. Viral infection with SARS-CoV-2-NanoLuc (Promega) was performed by diluting stock virus to an MOI of 0.5/ml in DPBS. Then, 100 μL of the diluted stock was added to each well for a final MOI of 0.05/well. Cells and viruses were incubated at 37° C. with 5% CO₂ for 1 hour, rocking the plates every 15 minutes. After this, the media was removed, 1 mL of prewarmed media with 1% FBS was added to each well, and the cells were incubated for 24 hours. The plate was then removed and allowed to cool to room temperature before 1 mL of prepared Nano-Glo Luciferase assay solution was added to each well and incubated for −3 min. After mixing by repeated pipetting, the lysate was transferred to a black luminance plate in quadruplicate and read using the luminescence endpoint protocol in a SpectraMax iD3 Multi-Mode Microplate Reader.

For the antibody-based dot blot assay, the MRC-5 cell line (Cat #CCL-171TM) was purchased from ATCC. The cells were cultured in the Eagle's Minimum Essential Medium (EMEM), including 10% fetal bovine serum (FBS) and Penicillin/Streptomycin. The cells were seeded at 1×10⁵ per well of 12-well plates for the inhibitory effects of fatty acids on coronavirus replication. When the cells were at around 70% confluent, the cells were incubated with human coronavirus HCoV-229E (ATCC, Cat #VR-740) in the 2% FBS EMEM for 1 hour at 370 C in the 5% CO₂ incubator. After an hour of viral incubation, the media was replaced with complete EMEM containing the appropriate dilution of drugs: Docosahexaenoic acid (Cat #90310, Cayman Chemical), Linoleic acid (Cat #90150, Cayman Chemical), α-Linoleic acid (Cat #90210, Cayman Chemical), Trichostatin (Cat #51045, Selleck Chemicals LLC), Depsipeptide (Cat #53020, Selleck Chemicals LLC), Valproic Acid (Cat #152064, MP Biomedicals), and Eicosapentaenoic acid (Cat #90110, Cayman Chemical), and then the cells were incubated an additional 48 hours. After 48 hours of drug treatment, the culture media were harvested and mixed with RIPA buffer to lyse the cells and release the virus. The lysates were loaded onto a pre-wetted nylon membrane assembled in a 96-well Manifold unit. After the vacuum filtration, the membrane was incubated with 5% fat-free milk for 30 minutes and subsequently incubated with rabbit anti-HCoV-229E antibody (Cat #40640-T62, SinoBiological) for 1 hour at room temperature and washed three times with TBS-T buffer, 10 minutes each. Then, the membrane was incubated with HRP-conjugated goat anti-rabbit IgG (H+L) secondary antibody (Cat #A16096, Invitrogen) for 1 hour at room temperature, washed three times with TBS-T buffer, 10 minutes each. The membrane was developed with Pierce ECL chemiluminescent substrate (Cat #32106, ThermoFisher). The Un-Scan-It software was used for analyzing the intensity of the dot blot, and the GraphPad Prism software was used for statistics and graphs.

In the case of oligonucleotide detection of HCoV-229E, the membrane was incubated with anti-HCoV-229E biotinylated-Oligo (5′-CCACTCTCAACAGCAAATACATTTTCTGAATAACCAACA (SEQ ID NO: 1) constructed with 3′ biotinylation) for 1 hour and washed with TBS-T 3×, 10 minutes each. The membrane was then incubated with Poly-HRP-Streptavidin (ThermoFisher Scientific, Cat #N200) in Poly-HRP dilution buffer (ThermoFisher Scientific, Cat #N500) for 30 minutes and washed with TBS-T 3×. The membrane was developed with ECL chemiluminescent substrate. Un-Scan-It software was used for analyzing the intensity of the dot blot, and GraphPad Prism 8.0 was used to plot the data.

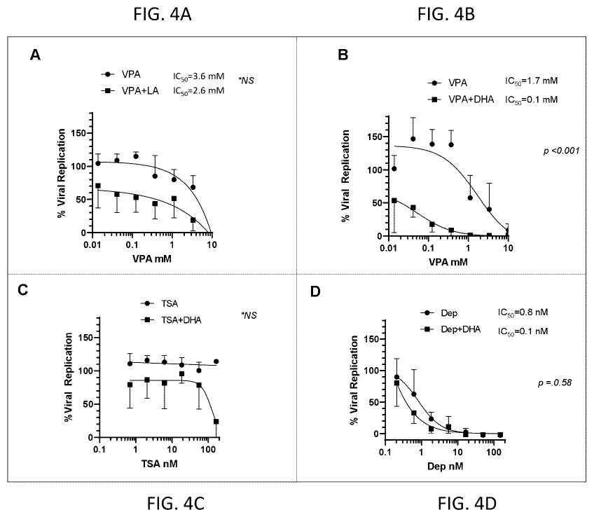

Impact of Fatty Acids and HDAC Inhibitors on Viral Replication of HCoV-229E. MRC5 cells were grown to a confluence of 70% and incubated with each of the following polyunsaturated fatty acids: docosahexaenoic acid (DHA), eicosapentaenoic acid (EPA), linoleic acid (LA), or alpha-linoleic acid (aLA)-obtained from Cayman Chemicals. The cells were incubated with HCoV-229E at an MOI of 0.1 for 1 hour, and then the drug was added to each cell in quadruplicate for each drug concentration. The cells were then harvested with Cell Lysis buffer (RIPA) as described herein, and lysate dotted into a 96-well manifold with a nitrocellulose filter for an antibody-based assay described herein. The assay was performed with a 1:3 dilution of the selected fatty acid starting at 100 μM. The IC₅₀ of each fatty acid was calculated using GraphPad Prism, using sigmoidal 3-factor curve fitting. Positive control of no drug was included and used for 100% viral replication. A negative control without any drug or virus was used for baseline subtraction. The IC₅₀ from a curve was accepted if the following conditions were met: (1) the R² of the fitted curve was above 0.65, (2) the standard error (SE) of the log(IC50) corresponded to a fold change smaller than 8, (3) the SE of the slope parameter was smaller than 8. Initial statistical analysis for comparing two dose-response curves was performed by two-way ANOVA with Tukey HSD, and the two curves were considered different when p<0.05. If two curves were considered different, a comparison of IC₅₀ was assessed using a statistical mixed-effect model to the log(IC50). Please note that in the case of Trichostatin A with and without DHA (FIG. 4C), the IC₅₀ could not be accurately calculated, and no attempt was made for statistical comparison.

RNA Processing and RNAseq Data Analysis. MRC-5 cells were grown to ˜70% confluency in complete media in 6-well plates, and then the culture media was replaced with serum-free media overnight. After this, the media was removed, and various drug combinations in complete media were added. For the time course study, 0.7 mM VPA was used, and cells were harvested every 24 hours for 96 hours. For the follow-up study, each well had one of the following conditions for 48 hours before harvesting: without HCoV-229E virus—media alone, 100 μM VPA, 500 μM VPA, 25 μM DHA, 100 μM VPA with 25 μM DHA, and 500 μM VPA with 25 μM DHA. With HCoV-229E virus—media alone, 500 μM VPA, 25 μM DHA, 100 μM VPA with 25 μM DHA, 500 μM VPA with 25 μM DHA, and 500 μM VPA with 25 μM DHA pre-incubated for 24 hours before viral infection. Cells were harvested by washing the cells twice with phosphate-buffered saline (ATCC), then trypsinizing the cells, neutralizing the trypsin with complete media, and centrifuging at 1500 rpm for 5 min. After removing the media, the cells were washed with PBS and spun down again. Finally, 500 μL of Trizol (Sigma) was added, and the standard protocol was followed to isolate RNA. RNA concentration was determined using UV-vis spectrometry at 260/280 nm in a Spectra-Max i3 plate reader (Molecular Devices).

Library preparation was completed on approximately 500 ng of total RNA using a standard workflow using the KAPA Stranded RNA-Seq Kit with RiboErase (KAPA Biosystems). rRNA was depleted, and then the remaining RNA was fragmented using divalent cations under elevated temperature and magnesium. The fragmented RNA was copied into double-stranded cDNA using random primers. Following this, adapters were ligated to the ends of the cDNA, and the product was amplified using PCR, which further purified and enriched the sequences for a final RNA-sequencing library. The libraries were quantified using a Qubit (ThermoFisher) and Bioanalyzer (Agilent). The libraries were pooled to 20 nM concentrations for sequencing. Sequences were read on a NextSeq 500 (Illumina) with 75 bp paired-end reads with an average of 35 million reads per sample. Raw data were demultiplexed to generate FASTQ files for analysis. Raw FASTQ files were uploaded to CLC Genomics Workbench 21 (Qiagen) for processing and initial analysis.

The provided RNA sequencing workflow was used to first quality control the raw data and remove any poor reads. Then the sequences were trimmed to remove the adapters. The sequences were then mapped to the Homo sapiens hg38 reference genome, and the gene count was generated. Gene counts were normalized to RPKM for differential expression analysis. Each treatment condition was compared to its respective control (media alone, with or without HCoV-229E virus) to examine changes in gene expression due to drug treatment. For differential expression analysis, the threshold for significance was set at over 1.5 fold change in expression and FDR p-value of less than 0.05. Differential gene expression was compared with CLC genomics set to pairwise expression comparing RPKM of one condition to the sham infected sham-treated MRC5 cells. The data was segregated into two groups. The group with the drug alone was compared to MRC5 cells without the drug. The group with drug and virus was compared to MRC5 cells infected with the HCoV-229E virus but no drug. Plots were made into NCCS comparing Log₂ (fold expression) between the two conditions in a 2-D scatterplot for the top 2000 differentially expressed genes. The gene expression data were processed through iDEP 1.9 and k-means cluster for 6 group clusters. The clusters were color-coded when plotted on the 2-D scatterplots to permit visualization of the impact on cluster groups by treatment.

Additionally, the % of viral RNA compared to total cellular RNA was determined by mapping the reads to the HCoV-229E viral reference genome (NCBI), and the total counts of viral RNA were determined and plotted as a percentage of total RNA sequences.

Time Course Data Analysis. After differential expression analysis, the total number of significantly up and down-regulated genes were plotted in GraphPad Prism. The gene list was then compared to the approximately 300 genes that interacted with SARS-CoV-2 genes per Gordon et al. (Gordon, D. E., et al., A SARS-CoV-2-Human Protein-Protein Interaction Map Reveals Drug Targets and Potential Drug-Repurposing. bioRxiv, 2020; and Gordon, D. E., et al., A SARS-CoV-2 protein interaction map reveals targets for drug repurposing. Nature, 2020. 583 (7816): p. 459-468) to determine which target genes could affect the virus. The log₂ (fold change) was plotted vs. the −log₁₀ (p-value) to create a volcano plot in NCSS software. The genes that met the significance criteria were mapped out in Cytoscape to visualize the virus-host protein interactions. Viral proteins were categorized relative to viral activity as viral assembly, replication, or pathogenicity. Western blot on select targets demonstrated host proteins to confirm the RNA-sequencing results at the protein level.

VPA and DHA RNAseq Study Data Analysis. Mapped data was uploaded to iDep v.94 (Ge, X., Methods Mol Biol, 2021. 2284: p. 417-443) and analyzed using its built-in algorithms and workflow. In summary, total differential gene expression was mapped in a heat map for the top 12,000 differentially expressed genes with at least 1.5 fold change relative to control and a p-value of 0.05. K-means clusters were generated for an appropriate number of clusters relative to the data and then plotted as a scatter plot in NCSS software. Principle components analysis (PCA) was also completed to examine how the samples were grouped in CLC Genomics (Qiagen, Inc). Differential expression analysis was done with DEG2, an FDR threshold p-value less than 0.05, and at least 1.5-fold change. Differential gene expression patterns were examined using a Venn diagram of unique and shared genes across samples. Finally, gene ontology (GO) analysis was performed using three categories: Biological Processes, Cellular Components, and Molecular Function. The GO analysis was further refined using PGSEA (Jambusaria, A., et al., BMC Bioinformatics, 2018. 19 (1): p. 217).

Western Blots. MRC-5 cells were grown to ˜70% confluence and then treated with valproic acid (MP Biomedical LLC) at concentrations between 0 and 5 mM for 24 or 48 hours. Protein was isolated by lysing cells in radioimmunoprecipitation buffer (Santa Cruz Biochemistry) for 1 hour, followed by centrifugation at 10,000×g for 15 minutes at 4° C. Protein concentration was determined using the Pierce BCA protein assay kit. For immunoblotting, proteins were separated using SDS-PAGE and transferred to nitrocellulose membranes. The membranes were blocked with 5% nonfat dry milk in Tris-buffered saline and then incubated with anti-PCNT (ab99341, Abcam), anti-DNMT1 (ab188453, Abcam), anti-BRD2 (ab139690, Abcam), anti-HMOX1 (ab52947, Abcam), and anti-GAPDH (G9545, Sigma). The membranes were incubated with appropriate horseradish peroxidase-conjugated secondary antibodies, and then bands were visualized by enhanced chemiluminescence. Densitometric analysis was performed using Un-Scan-It 7.0 software (Silk Scientific).

Optum Dataset Analysis. Data from The Optum data set representing more than 3 million patients were collected (state the dates of inclusion of the study) for patients undergoing COVID-19 testing. The data are analyzed as both a case-control and retrospective cohort study. The patients were then tested for COVID-19 by a nucleic acids method. Variables used for adjustment and matching were gender, age, race, ethnicity, insurance, vaccine status, cancer, cancer treatment, CHF, COPD, renal disease, DM, region of the country where the patient was tested, and the calendar trimester when the patient was tested. The central hypothesis tested was the association with detection of COVID-19 by nucleic acid testing in patients who have documentation of valproic acid use in their medication list. The null hypothesis (Ho) was that SARS-CoV2-test positivity has no association with VPA therapy. The secondary study is a retrospective cohort study conducted with multivariate analysis, using Logistic regression for the categorical variables. The null hypothesis (Ho) for this portion of the analysis is that SARS-CoV2 test positivity has the same hazard ratio for 30-day all-cause ER admission regardless of VPA use and that SARS-CoV2 test positivity has the same hazard ratio for 30-day all-cause hospital admission regardless of VPA use. Frequency distribution of the patient demographics and prior medical history was performed for gender, race, ethnicity, geography, and comorbidities. Notably, the patients treated with VPA had higher levels of AIDS, CHF, COPD, Cerebral vascular disease, dementia, diabetes, prior MI, and peripheral vascular disease. As these conditions are known to predispose to a higher risk of morbidity from SARS-CoV2, a matched case-control model was created in which patients without listed VPA use were matched for comorbidities including AIDS, CHF, CPOD, CVD, dementia, diabetes, prior MI and PVD using a propensity control model. Comparisons between the VPA and non-VPA groups were performed using Logistic regression.

Valproic Acid Levels in a National Data Set. The serum VPA levels tested during the third week of June 2021 were provided kindly by LabCorp. Data were analyzed by gender, with similar distributions, and separated by quartiles for comparisons. No attempt was made to match patients between the LapCorp cohort and the Optum cohort, as both groups were fully de-identified. No information on the dosing of valproic acid is known for the patients studied from either cohort (Optum or LabCorp).

NGS for Drug Inhibition of HCoV-229E replication. Differential gene expression analysis was done in two groups. The first group (group 1) were MRC5 cells in which the cells were treated with VPA, DHA, VPA+DHA, or control (no drug). The reference control for the differential expression was the expression of MRC5 without a drug. The second group (group 2) were MRC5 cells in which the cells were treated with the HCoV-229E virus, then drug for 48 hours, and harvested for RNA as described in the methods section for RNASeq. The control group was MRC5 cells treated with HCoV-229E virus without drug, and an additional set of cells was pre-incubated with 0.5 mM VPA+25 μM DHA for 24 hrs prior to HCoV-229E incubation (the most robust condition for inhibition of viral replication). The group 2 cells had their gene expression referenced to the MRC5 incubated with virus only (no drug). The RNA was run for RNASeq as described herein and then analyzed relative to each group control. Shared genes for the conditions, including DHA, VPA, or DHA+VPA, were assessed for differential gene expression in both the drug-treated and the drug-treated/virus-treated groups. Differential gene expression was then assessed under VPA, DHA, and DHA+VPA conditions and screened for p<0.05, with a gene expression cutoff of 1.5 fold relative to control MRC5 or control MRC5 infected with HCoV-229E. Hierarchical clustering was performed with dual dendrogram heat maps plotting absolute fold gene expression with Z-scale normalization (to provide linear clustering but symmetric expression around 0 for induction and repression).

Treatment of MRC5 cells with DHA and VPA prior to infection with SARS-CoV2 to inhibit replication. In the experiment to determine if pretreatment of MRC-5 cells with drug would inhibit replication of SARS-CoV2 virus, MRC-5 cells were grown in the presence of DHA (25 μM), VPA (0.5 mM), or DHA and VPA (25 μM and 0.5 mM respectively) or media not containing drug, for 4 days before plating 300,000 pretreated cells into 6 well plates in the appropriate drug containing media. The following day, SARS-Cov2 virus was added to each well at a MOI of 0.1, with no virus acting as a control. Media and virus was removed after 1 hour and was replaced with media containing the appropriate drug. After incubating the cells for 24 hours, media was removed and RNA harvested from the cells using Trizol. RNA was analyzed for SARS-Cov2 RNA by real time RT-PCR. This experiment was repeated, and also included a 3 day pretreatment with the drugs before viral infection and performed in 12 well plates with 80,000 cells per well. RNA was then analyzed by Next Gen Sequencing.

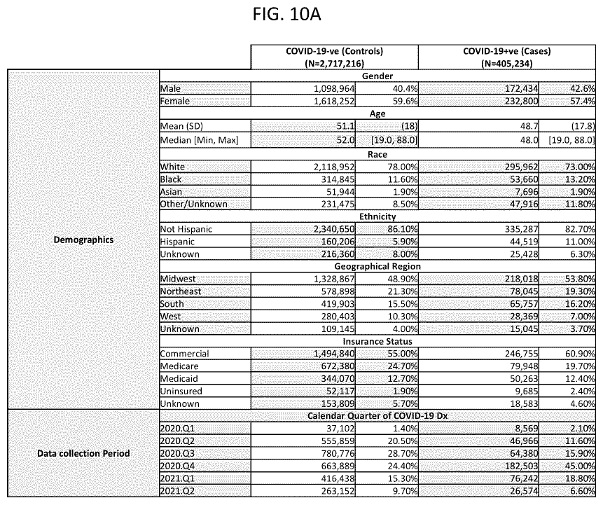

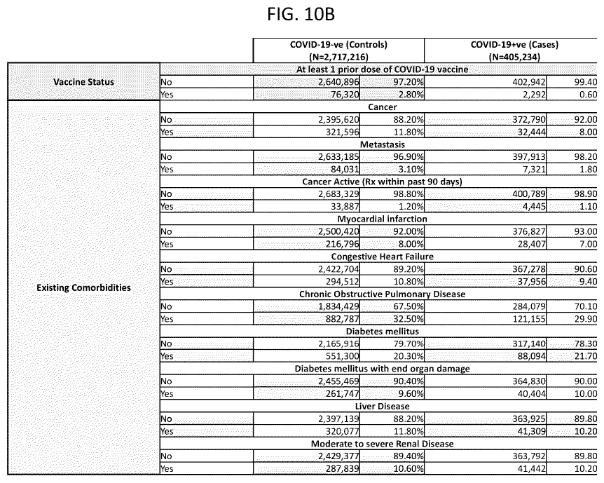

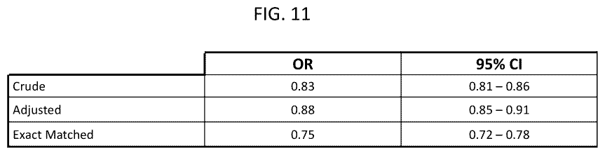

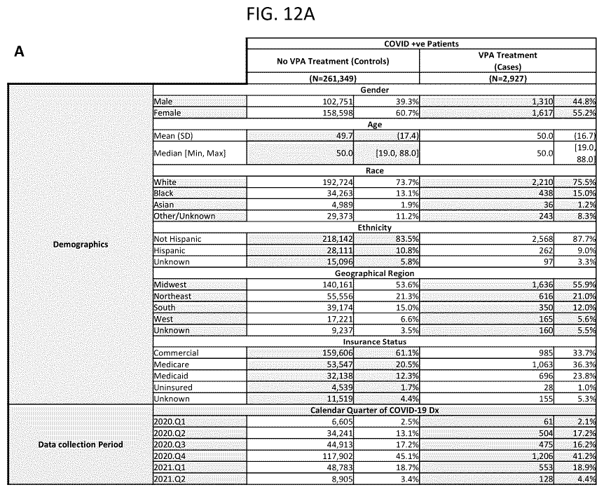

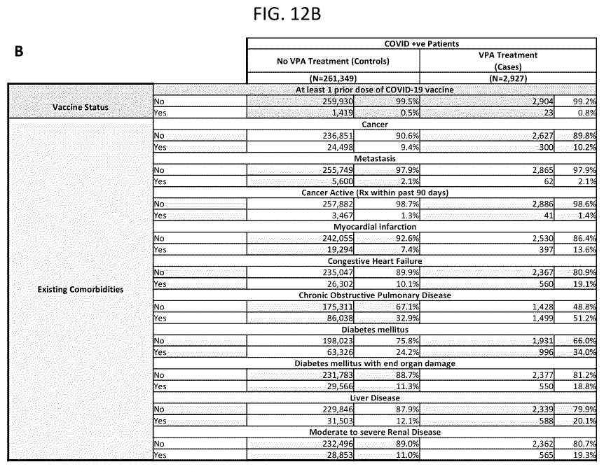

Results. Case-Control Analysis of the Optum Dataset. FIG. 10 shows the characteristics of patients in the Optum dataset listed as having been prescribed VPA in the retrospective cohort of patients tested for COVID-19. The Optum dataset collected information from more than 3 million patients tested for COVID-19 using nucleic acid testing from the first quarter of 2020 until the second quarter of 2021. As shown in FIG. 10, more than 400,000 patients tested positive for COVID-19, and 2.7M patients tested negative for a net positivity rate of 14.9%, with a similar distribution between males and females-though females were tested significantly more often than males (1.46 fold). The age distributions between males and females were comparable between COVID+ and COVID-patients. At the time of this COVID-19 testing, significant geographic differences were not observed between COVID+ and COVID-patients. Race and ethnicity patterns did demonstrate higher percentages of black and Hispanic patients in the COVID+cohort, consistent with other previously reported statistics (Chang, M. H., et al, J Public Health (Oxf), 2022. 44(2): p. e211-e220; Wong, M. S., et al., Int J Environ Res Public Health, 2021. 18(9); Nguyen, L. H., et al., Racial and ethnic differences in COVID-19 vaccine hesitancy and uptake. medRxiv, 2021; and Kopel, J., et al., Front Public Health, 2020. 8: p. 418). FIG. 11 shows the distribution of positive and negative patients over the study period, from the first quarter of 2020, until the second quarter of 2021. The peak of the test positivity was in the fourth quarter of 2020, representing 45% of the positive cases in the study period. The insurance status between COVID+ and COVID− patients was similar over the study period. Importantly, >97% of the COVID+ and COVID− patients had no prior immunization at the time of testing. This finding permits an evaluation of the potential impact of VPA use without the confounding impact of prior immunization. FIG. 12 provides the characteristics of patients prescribed or not prescribed VPA in the retrospective cohort of COVID-19 infected patients. FIG. 12 shows the odds ratios for univariate and multivariate analyses of the likelihood of test positivity when patients have been “exposed” to a VPA prescription, as determined by their active medications list. Actual serum VPA measurements were unavailable for this patient cohort during the COVID-19 testing.

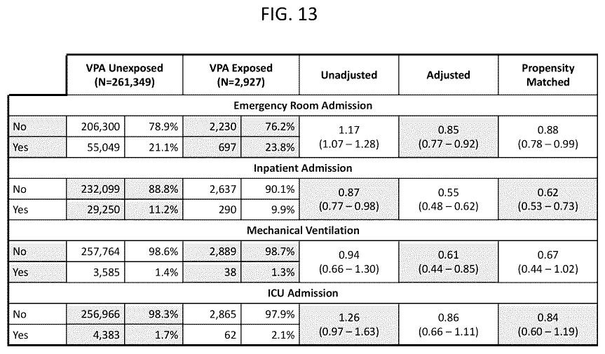

FIG. 13 provides the clinical progression of COVID-19 in patients prescribed compared to patients not prescribed VPA in the COVID-19 infected cohort.

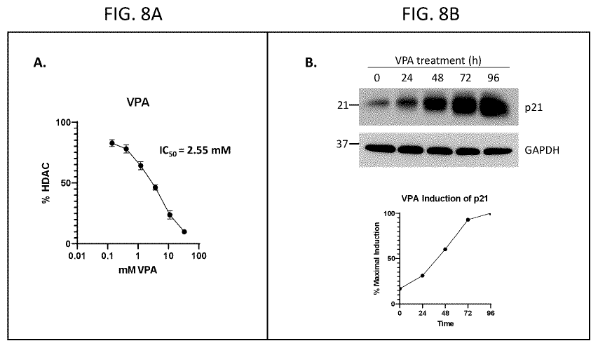

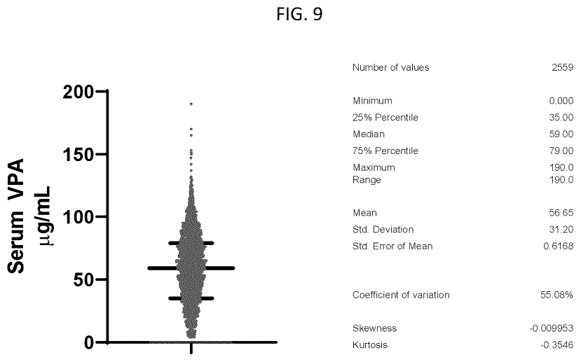

As a control, a sampling of the VPA levels collected during two weeks in June 2021 is shown in FIG. 8. The distribution of serum VPA levels demonstrates a mild platykurtic near-normal distribution of values with skewness of −0.009953 and kurtosis of −0.3546, with a mean and median of 56.7 μg/mL and 59.0 μg/mL, respectively. FIG. 8 shows that fewer than 25% of the patients tested had a serum level of 80 μg/mL (˜0.55 mM). Most patients on VPA for a seizure disorder have target concentrations of 50-100 μg/mL, with enhanced toxicity found at levels>125 μg/mL. If less than 50% of the patients on VPA have achieved therapeutic levels (assuming they are the same between seizure disorder and COVID+protection), then the OR found in FIG. 12 may underestimate the protective effect by more than two-fold. The association of VPA use with a decreased rate of positive COVID-19 testing does not necessarily demonstrate causality. However, these findings are consistent with the possible role of VPA in diminishing the rate of COVID-19 and the severity of the disease among those who test positive. Also shown in FIG. 12 is the distribution of patients testing positive for COVID-19 amounts in the VPA+ and VPA− groups for age, race, and geography. VPA+ and VPA− patients had similar distributions for these three variables.

Logistic regression of a univariate (unadjusted) comparison of patients “exposed” to VPA compared to VPA-negative patients shows no protective effect against emergency room visits; however, when either a propensity-matched model or a multivariate-adjusted model is applied, controlling for comorbidities such as diabetes, heart disease, congestive heart failure, and chronic obstructive pulmonary disease, then significant reductions were seen of 12-15% in ER visits, 17-45% reduction in hospital admission, 33-39% reduction in mechanical ventilation, and 14-16% reduction in ICU admissions. These findings show that patients who actively take VPA are less likely to develop COVID-19, and when they do develop this infection, they are less likely to require an emergency department visit, hospitalization, ICU admission, or mechanical ventilation.

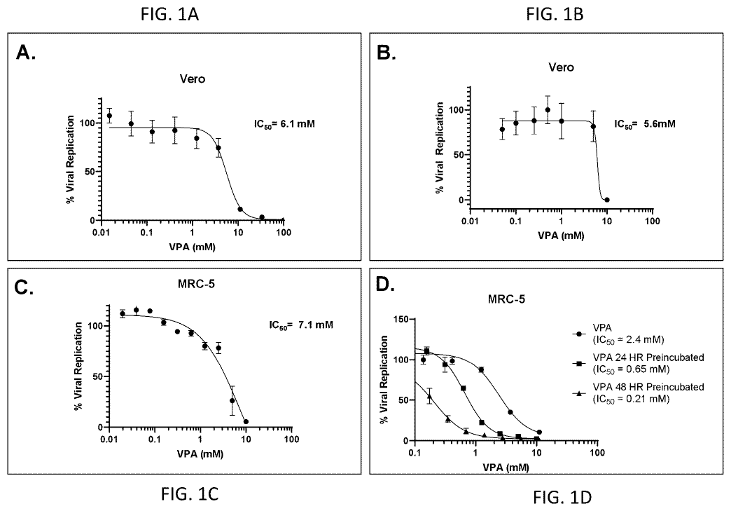

VPA Inhibition of SARS-CoV2 Viral Replication. Since the epidemiological data demonstrate a putative role for VPA inhibition of SARS-CoV2 activity, the in vitro activity of VPA was assessed in more detail. To determine the optimal dosing for an effect of VPA in viral replication, the IC₅₀ of VPA in directly inhibiting HDAC2 activity in Vero cells was measured (FIG. 8A) and determined it to be at least 2.5 mM—a level almost least threefold higher than accepted toxicity thresholds in humans. As p21 induction is a common consequence of HDACi, the time course for induction was evaluated and it was found that maximal induction occurs at 72 hours, with the earliest induction seen at 24 hours. Since the initial models of VPA inhibition of SARS-CoV2 predicted a direct inhibition of HDAC2, the viral inhibition assays were bracketed around this IC₅₀ (FIGS. 1A-D). Viral inhibition was measured by an antibody-based assay (FIG. 1A), Firefly Luciferase assay (FIG. 1B), and by oligonucleotide hybridization-based assay (FIGS. 1C and D) with similar results for both SARS-CoV2 (FIG. 1A and FIG. 1B) and HCoV-229E (C). However, preincubation with VPA for 24 or 48 hours reduces the IC₅₀ for inhibition of viral replication by more than 3-fold (FIG. 1D), bringing the dose range for VPA within therapeutic limits. As SARS-CoV2 is pathogenic and requires specialized facilities for manipulation, the impact of VPA on viral replication on the related alpha-coronavirus HCoV-229E (Hierholzer, J. C., Virology, 1976. 75 (1): p. 155-65), thought to be responsible for many cases of the “common cold” (Pohl-Koppe, A., et al., Detection of human coronavirus 229E-specific antibodies using recombinant fusion proteins. J Virol Methods, 1995. 55 (2): p. 175-83) was further studied. HDAC inhibition is an effective mechanism for altering gene expression patterns towards a more differentiated state and has been used as an approach for several malignancies (Gottlicher, M., Ann Hematol, 2004. 83 Suppl 1: p. S91-2; and Xia, Q., et al., Cancer Res, 2006. 66(14): p. 7237-44). When used as a cancer therapeutic, the effect of VPA is also delayed, as it takes time for gene expression patterns to manifest cellular changes (Gottlicher, M., Ann Hematol, 2004. 83 Suppl 1: p. S91-2). Chronic administration of VPA, for instance, in prostate cancer cell lines can cause significant cellular toxicity and alterations in androgen receptor levels at much lower doses than observed during acute administration (Xia, Q., et al., Cancer Res, 2006. 66(14): p. 7237-44).

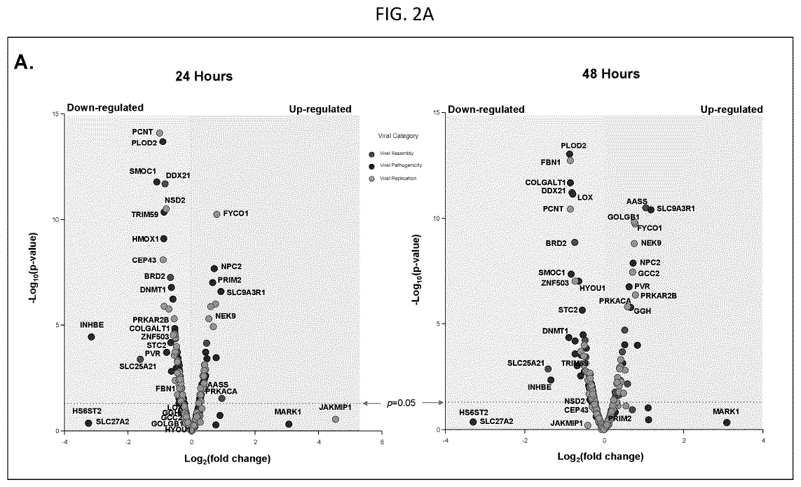

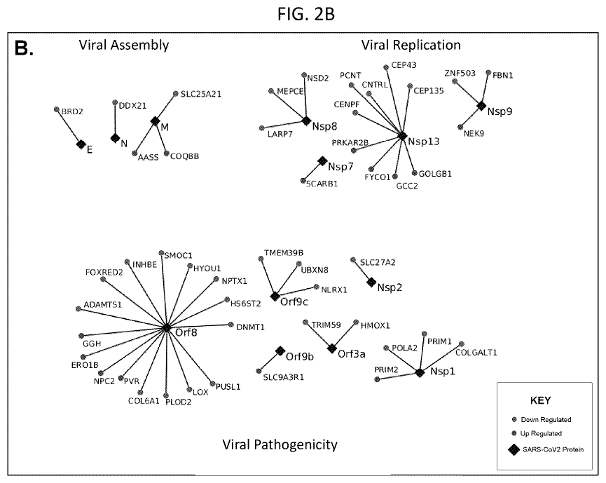

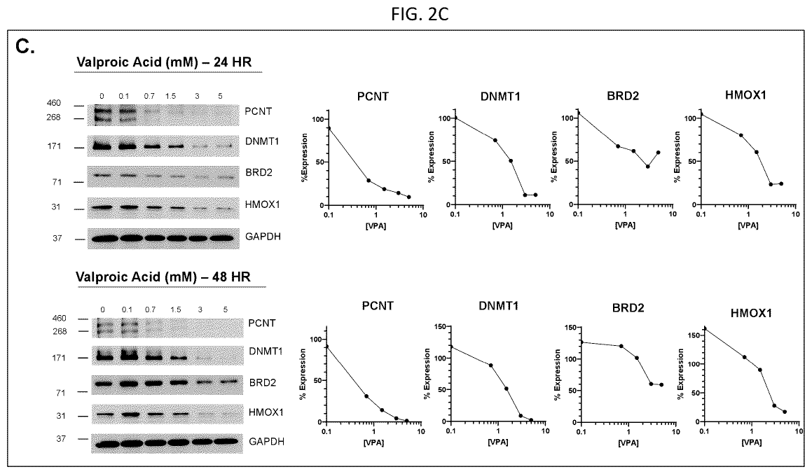

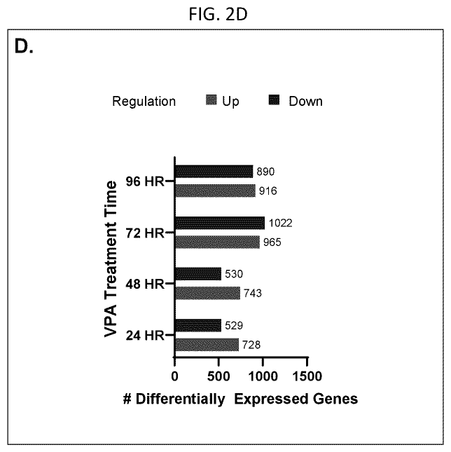

In order to help predict the impact of VPA gene expression changes, RNAseq was performed on the hCoV-229E permissive MRC5 cells treated with VPA for 24 and 48 hours and assessed the impact on the expression of the “Gordon” gene set. FIGS. 2A and B show volcano plots of the 300 gene set, demonstrating that at 24 and 48 hours, there are changes in gene expression with both down and up-regulated genes. The overall impact is more down-regulated than up-regulated genes, with a significant difference in the down-regulation of genes involved in viral replication, as defined by Gordon et al. (Gordon, D. E., et al., A SARS-CoV-2-Human Protein-Protein Interaction Map Reveals Drug Targets and Potential Drug-Repurposing. bioRxiv, 2020; and Gordon, D. E., et al., Nature, 2020. 583 (7816): p. 459-468). Both viral pathogenicity and replication genes are down-regulated preferentially with VPA treatment. Confirmation of the gene expression was performed by Western blot for PCNT, DNMT1, BRD2, and HMOX1 at 24 and 48 hours, consistent with the gene expression data (FIG. 2C). Overall gene expression with VPA treatment demonstrated significant differences in up and down differentially regulated genes after 48 hours, with a predominance of up-regulated genes at 24 and 48 hours (FIG. 2D).

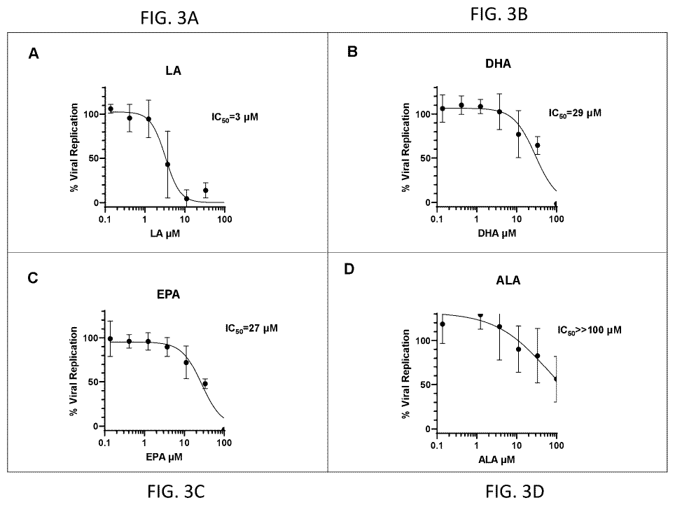

Effect of Polyunsaturated Fatty Acids (PUFAs) on HCoV-229E Viral Replication., Polyunsaturated fatty acids (PUFAs) were tested in models of HCoV-229E viral inhibition. The omega-3 fatty acids EPA (eicosapentaenoic acid), ALA (alpha-linolenic acid), and DHA (docosahexaenoic acid) were tested as well as the omega-6 fatty acid LA (linoleic acid) for their effect on HCoV-229E viral replication in MRC5 cells. Cells were incubated with HCoV-229E for one hour at an MOI of 0.1, and media containing varying amounts of the PUFAs were added. The cells were incubated for another 48 hours before harvesting and assessing for viral replication using an antibody-based assay. As shown in FIG. 3A, LA had a substantial inhibitory effect on HcoV-229E (Sofia, F. O. A., et al., Comput Struct Biotechnol J, 2022. 20: p. 139-147; Toelzer, C., et al., Science, 2020. 370(6517): p. 725-730; Toelzer, C., et al., Sci Adv, 2022. 8(47): p. eadc9179; and Goc, A., et al., Sci Rep, 2022. 12(1): p. 19114). Surprisingly, DHA (FIG. 3B), and EPA (FIG. 3C) also demonstrated significant inhibition of viral replication, while ALA demonstrated the least impact. As the intended use of PUFAs was a combination of VPA and DHA, the combination of VPA with both DHA and LA was tested to determine if the combination was mutually antagonist, additive, or supra-additive (e.g., synergistic). MRC5 cells were pre-incubated with VPA with and without LA or DHA at a fixed concentration of 25 μM. While 25 μM LA inhibited viral replication (FIG. 4A), as expected, it did not significantly improve the IC₅₀ of VPA in the inhibition of viral replication, as the 95% confidence limits were grossly overlapping. In contrast, the combination of 25 μM DHA+VPA resulted in a substantial shift in the VPA IC₅₀, with nearly a 10-fold improvement in the inhibition of viral replication (FIG. 4B), with a two-sided ANOVA of p<0.001 and completely non-overlapping 95% confidence limits for the IC₅₀. Please note that while the IC₅₀ of VPA was higher in the LA combination experiment than the DHA combination when the two VPA-only curves were compared across the two experiments (two-tailed nested t-test), they were not statistically different (p=0.41). The differences were consistent with known interassay variability in the measurements of viral replication.

Next, it was tested whether the VPA+DHA combination was exclusive to VPA or represented a broader phenomenon present with other HDAC inhibitors. Depsipeptide and trichostatin were assessed for the impact of the combination with and without 25 μM DHA on viral replication. FIG. 4C shows that Trichostatin A alone did not significantly inhibit viral replication, and the combination with DHA causes inhibition across the board, as expected. However, the inhibition of replication was augmented at the very highest dose of Trichostatin A tested. In contrast, depsipeptide demonstrated significant inhibition of viral replication; however, DHA shifted the IC₅₀ from 0.8 to 0.1 nM but was not significant by two-tailed ANOVA (p=0.58). While trichostatin and depsipeptide inhibit HDAC1 and HDAC2, trichostatin treatment of multiple cell lines has demonstrated a distinctly different gene expression pattern than depsipeptide (Chang, J., et al., Br J Cancer, 2012. 106 (1): p. 116-25). This differential activity may explain why VPA and depsipeptide have better antiviral activity as a monotherapy. Trichostatin's inhibition of HDACs is a direct effect through chelation of zinc in the active site, preventing histone unpacking, while VPA and depsipeptide exert their inhibition more directly on the enzymatic activity of deacetylation, which may have activity more broadly than just acetylated histones in the nucleus (Marks, P. A., et al., Curr Opin Oncol, 2001. 13 (6): p. 477-83). However, regardless of this differential activity in gene expression, the combination with DHA shows a substantial synergy of antiviral activity.

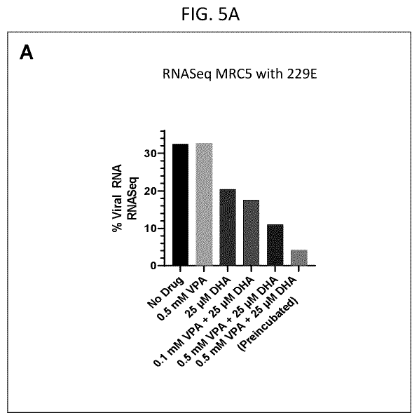

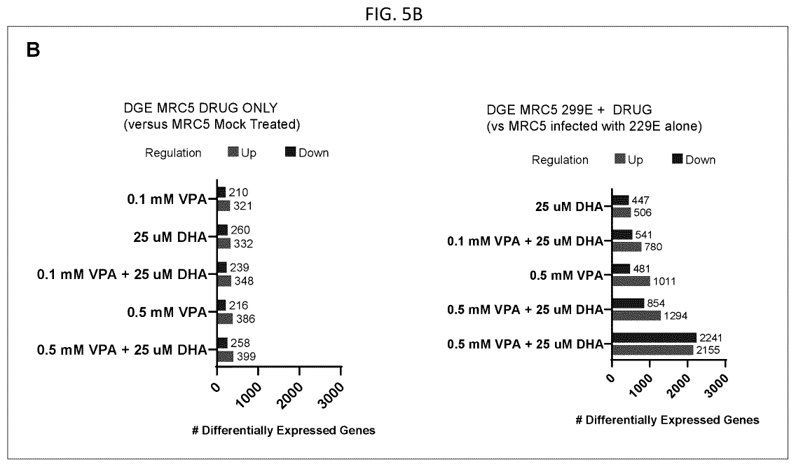

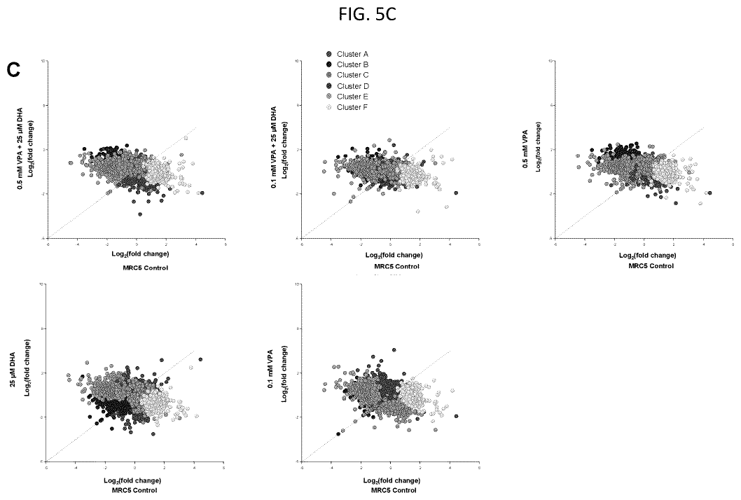

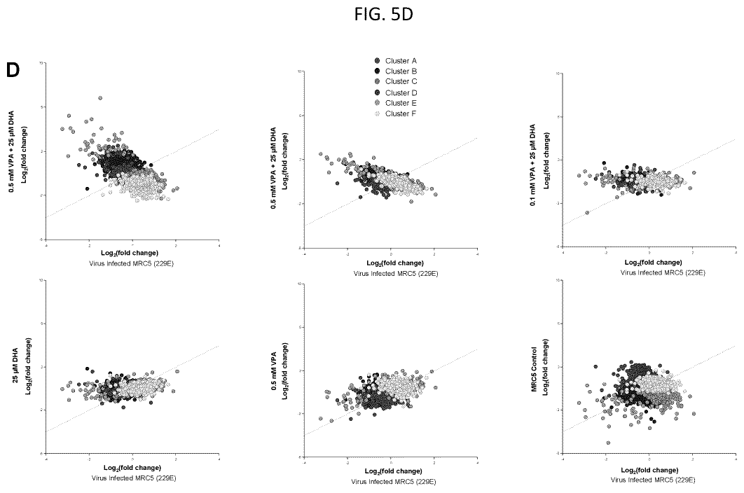

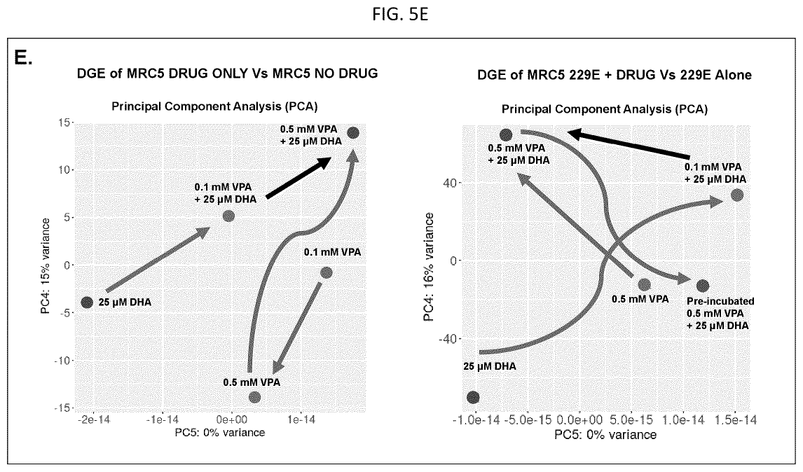

Gene Expression of MRC5 Cells is Dramatically Altered by HCoV-229E Infection when Treated with VPA+DHA. It was tested whether DHA impacted VPA-regulated target gene expression or HCoV-229E viral replication. Twelve experiments were performed with the MRC5 cells, either in the presence or absence of drug (VPA or DHA or both) and virus (HCoV-229E). The virus was added at an MOI of 0.1 for one hour, and the drug (VPA, DHA, or both) was added after the viral incubation for 48 hours before RNA was harvested. In one experiment, MRC5 cells were “pre-incubated” with 0.5 mM VPA+25 μM DHA for 24 hours before adding the HCoV-229E virus and then the drug combination continued for another 48 hours before RNA harvest. RNA from the cell lysates were analyzed for gene expression by RNAseq on an Illumina HiSeq 2000. Viral replication was determined by the amount of viral RNA detected expressed as the % of Total RNA from each experimental condition. As seen in FIG. 5A, viral replication was not significantly impacted when VPA was added during viral incubation. In contrast, 25 μM DHA added immediately after viral incubation resulted in more than a ⅓ drop in viral RNA, consistent with the viral replication experiments. The contemporaneous addition of 0.1 mM VPA and 0.5 mM VPA with 25 μM DHA resulted in a further reduction of viral RNA, but the most pronounced effect was the combination of DHA and VPA with 24-hour preincubation.

Differential gene expression (DGE) was performed using iDEP 0.96 (Ge, X., Methods Mol Biol, 2021. 2284: p. 417-443) to identify the top 2000 genes in each treatment group when compared to either untreated MRC5 cells, or MRC5 cells treated with virus without drug. In the case of the drug along group, 1914 genes were differentially expressed, which was reduced further to between 500-650 differentially expressed genes when screened by a p-value of <0.05 (FIG. 5B) in the drug-treated group. A substantial differential gene expression pattern was observed due to viral infection in the second group treated with HcoV-229E (FIG. 5B). K-means clustering was performed to identify six major gene clusters, and a scatterplot was made comparing the Log₂ (fold change) expression for each treatment group versus their control group, as shown in FIGS. 5C and D. Notable in FIGS. 5B and 5D, the preincubation group of 0.5 mM VPA+25 μM DHA treated for 24 hours prior to viral infection, resulted in the most profound DGE. The Pearson correlation coefficient in FIG. 5D starts at near 0 for the control MRC5 cells versus virus-infected cells for the 6 clusters and continues to be slightly positive in VPA and DHA-treated cells; however, when the combination of VPA and DHA is used, the slope is distinctly negative with the most profound change in the pre-incubated experiment, where the Pearson correlation coefficient is −0.7 (Table 1). Visual confirmation of these substantial gene expression changes is noticeable in the color-coded clusters where Cluster B, C, and F significantly increase their gene expression in the pre-incubated drug combination relative to control. In contrast, Cluster A and E have fewer relative gene expression changes. These findings indicate that hundreds of genes are impacted in their response to HCoV-229E infection when pre-incubated with VPA and DHA in a manner that does not occur with either drug alone.

Table 1. Pearson correlation coefficient values and 95% confidence interval for the drug-alone group (top) and virus-infected plus drug (bottom) under the various tested treatment conditions. The combination of VPA and DHA, in the pre-incubated experiment showed the most profound change with the Pearson correlation coefficient of −0.7.

| | Pearson's CC | 95% Confidence | | DOF = 1998 |
|---|---|---|---|---|
| Treatment Group | r | Lower | Upper | p-value |

DRUG ALONE GROUP

| | | | | |
|---|---|---|---|---|
| 0.1 mM VPA | −0.2706 | −0.3107 | −0.2295 | <0.0001 |
| 25 μM DHA | −0.2636 | −0.2223 | −0.3039 | <0.0001 |
| 0.5 mM VPA | −0.3956 | −0.3579 | −0.4319 | <0.0001 |
| 0.1 mM VPA + 25 μM DHA | −0.3279 | −0.2882 | −0.3664 | <0.0001 |
| 0.5 mM VPA + 25 μM DHA | −0.4155 | −0.3785 | −0.4510 | <0.0001 |

VIRUS INFECTED + DRUG

| | | | | |
|---|---|---|---|---|
| MRC5 Control | −0.0843 | −0.0406 | −0.1276 | 0.0002 |
| 0.5 mM VPA | 0.5308 | 0.4985 | 0.5615 | <0.0001 |
| 25 μM DHA | 0.3572 | 0.3183 | 0.3948 | <0.0001 |
| 0.1 mM VPA += 25 μM DHA | −0.0752 | −0.0314 | −0.1186 | 0.0008 |
| 0.5 mM VPA + 25 μM DHA | −0.6242 | 0.5966 | −0.6501 | <0.0001 |
| Preincubated 0.5 mM VPA + 25 | −0.7250 | −0.7035 | −0.7452 | <0.0001 |
| μM DHA | | | | |

To better understand the overall impact of the various drug combinations on MRC5 gene expression, Principle Component Analysis was performed for the Differential Gene Expression to achieve a two-dimensional plot, representing the totality of the gene expression of each condition (e.g., dimensionality reduction) as shown in FIG. 5E. In the drug-treated group, treatment with either 25 μM DHA or 0.1 mM VPA resulted in two relatively independent gene expression profiles which were further impacted by the addition of either more VPA or the addition of VPA to the DHA, culminating with the most significant difference found by the combination of 0.5 mM VPA+25 μM DHA. Similarly, when the virus is added, both the DHA and VPA-alone patterns are distinctly different, converging with the combination and the pre-incubated combination providing the most significantly different condition. These findings indicate that treatment with DHA is a modifier of gene expression, not just binding to the virus in a hydrophobic pocket to affect uptake. Also, these results indicate that when virus infection occurs, the gene expression patterns of DHA and VPA are independent, and the pre-incubated combination is also substantially different than either alone, demonstrating a form of synergy.

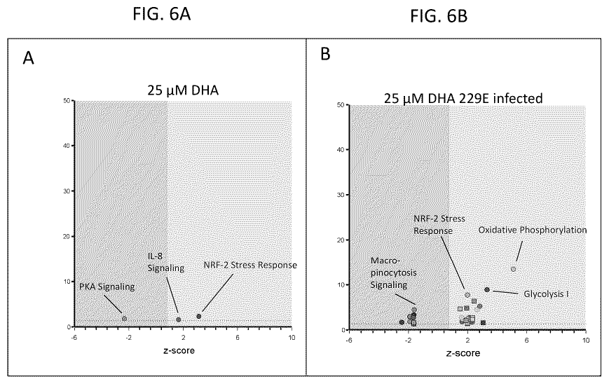

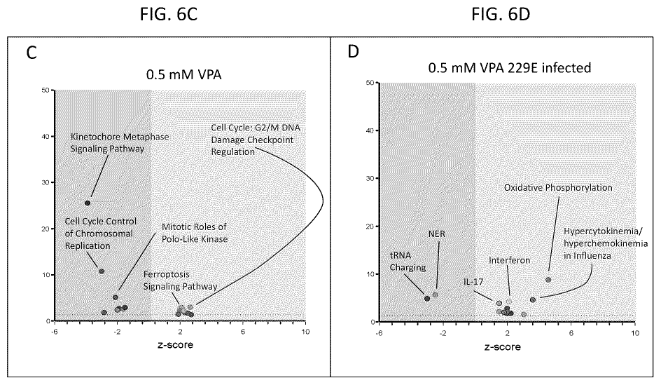

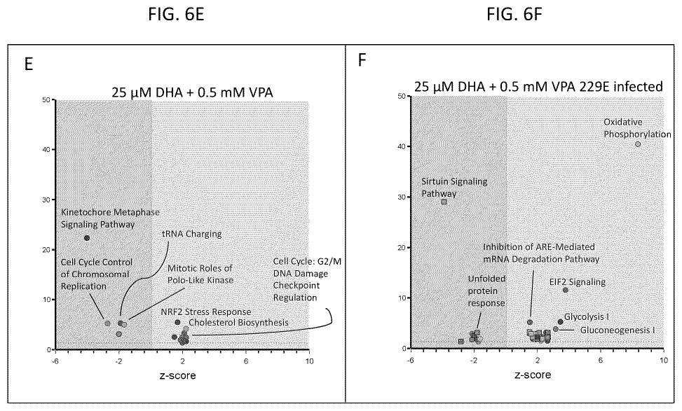

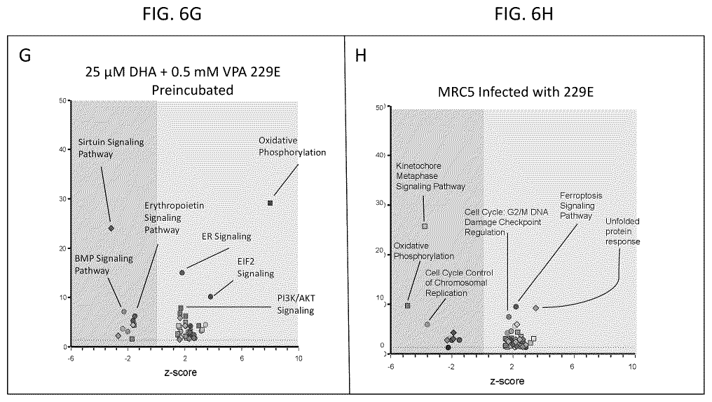

It was also assessed which molecular pathways are most impacted by the treatment with DHA and VPA. The Qiagen Ingenuity Pathway Analysis (IPA) was used. The RNASeq data on MRC5 cells were entered into IPA and subjected to pathway analysis, with output including pathway, relative Z-score, and p-value. Data were plotted on a volcano plot, as shown in FIGS. 6A-F, with each condition labeled, pairing each drug condition with drug along versus drug+virus. In the case of 25 μM DHA, viral replication induced positive Z-scores for oxidative phosphorylation, glycolysis and NRF-2 stress response, and a negative Z-score for micro-pinocytosis signaling. VPA treatment alone (FIGS. 6C and 6D) induced pathways commonly associated with HDACi in general, but inflammatory pathways became dominant with the addition of the virus. The combination of the virus with 25 μM DHA+0.5 mM VPA demonstrated a further induction of oxidative phosphorylation and enhanced energy metabolism but also showed a substantial down-regulation of sirtuin signaling (FIG. 6F), which is continued with preincubation (FIG. 6G). The genes associated with each pathway are shown in Table 2. These gene expression changes demonstrate a strong antiviral response induced by the combination of an HDACi and DHA. This antiviral effect is maintained even with doses of VPA which otherwise would not protect in the absence of DHA.

TABLE 2
Table of different genes associated with the respective pathways in IPA.
Pathways/Genes Prior to Viral Infection

| | |
|---|---|
| NRF-2 Stress Response | ACTA2, ATF4, CBR1, CYP2U1, DNAJB5, EPHX1, |
| | FOSL1, FTH1, FTL, GSR, GSTA4, GSTM2, GSTM3, |
| | HMOX1, HSPB8, MAPK3, MAPK7, MAPK9, |
| | MGST2, NQO1, NQO2, PIK3CD, PIK3R3, |
| | PMF1/PMF1- |
| | BGLAP, PRDX1, SCARB1, SQSTM1, TXN, |
| | TXNRD1 |
| Kinetochore Metaphase Signaling | CDC26, CENPT, CENPW, H2AC18/H2AC19, |
| Pathway | MAD2L2, MASTL, |
| | PPP1R14B, PPP2R5D, STAG2 |
| Cell Cycle Control of Chromosomal | CDC6, CDK1, CDK15, CDK17, CDK5, CDT1, |
| Replication | DBF4, MCM2, MCM4, |
| | MCM5, MCM6, MCM7, PCNA, POLA1, RPA1, |
| | TOP2A |
| Mitotic Roles of Polo-Like Kinase | CCNB1, CCNB2, CDC20, CDK1, FBXO5, KIF11, |
| | KIF23, PLK1, |
| | PPP2R5B, PRC1, STAG2 |
| Cell Cycle: G2/M DNA Damage | AURKA, CCNB1, CCNB2, CDK1, CKS1B, PLK1, |
| Checkpoint Regulation | TOP2A |

Pathways/Genes After Viral Infection

| | |
|---|---|
| Macro-pinocytosis Signaling | ITGB8 |
| Nucleotide Excision Repair Pathway | POLR2A, POLR2J2/POLR2J3, RPA1 |
| Hypercytokinemia/ | AREG, CCL2, CXCL8 |
| hyperchemokinemia in influenza | |
| Interferon | IFI6, IFIT1, IFIT3, IFITM1, IFNAR2, IRF1, ISG15, |
| | MX1 |
| IL-17 | CCL2, MAPK3, MAPK9, PIK3CD, PIK3R3, SRSF1 |
| Sirtuin Signaling Pathway | AGTRAP, ATP5MC1, CXCL8, CYC1, G6PD, |
| | GABARAP, GABPA, GADD45B, H1-1, H1-5, |
| | H3C14, H4C11, MAP1LC3A, MAP1LC3B2, |
| | MAPK3, MAPK7, MT-ND3, MT-ND6, NBN, |
| | NDUFA2, NDUFA7, NDUFB7, NDUFB9, NQO1, |
| | PCK2, PFKM, POLR1E, RBBP8, RRP9, |
| | SIRT1, TIMM10, TIMM44, TSPO |
| Inhibition of ARE-Mediated mRNA | EXOSC6, EXOSC8, MAPK3, MAPK7, PPP2R5D, |
| Degradation Pathway | PSMB10, PSMB9, |
| | TNFRSF11B, ZFP36L1, ZFP36L2 |
| EIF2 Signaling | ACTA2, ATF4, EIF5, MAPK3, PIK3CD, PIK3R3, |
| | TRIB3 |
| Unfolded protein response | CEBPG, DDIT3 |

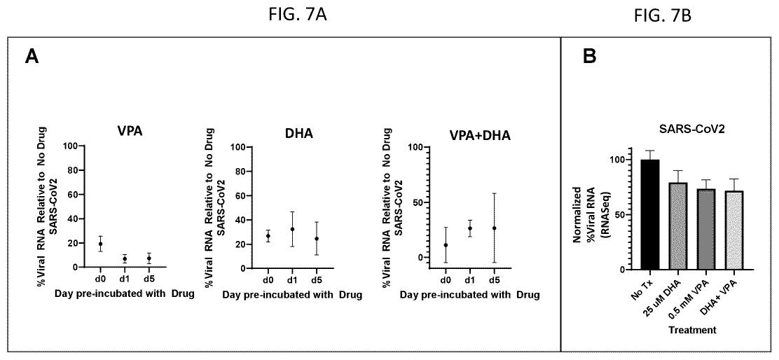

VPA+DHA Treatment of SARS-CoV2 Inhibits Viral Replication. It was tested whether the antiviral activity of DHA+VPA was present in SARS-CoV2. MRC5 cells were treated with SARS-CoV2 at an MOI of 0.1, and treated with VPA, DHA, DHA+VPA or control MRC5 without drug. SARS-CoV2 RNA was then detected by qRT-PCR (FIG. 7A). Inhibition of viral replication was assessed by plotting the % viral RNA detected compared to the control MRC5 cells without drug. As can be seen in FIG. 7A, VPA, DHA and the combination of VPA+DHA each have substantial inhibitory activity against SARS-CoV2 at the doses tested, with preincubation by 24 hours dropping the viral inhibitory effect from 20% to less than 10% (i.e., more than 50% reduction). Additional preincubation up to 5 days did not provide any additional effects. DHA conferred an inhibitory effect by itself, as it did with HCoV-229E, but preincubation did not improve the effect. Finally, the combination of VPA+DHA was highly effective at d0. MRC5 does not have the canonical receptor ACE2; hence, viral binding, uptake and spreading are markedly diminished in MRC5 cells. It was estimated that less than 1% of the total isolated RNA was SARS-CoV2, consistent with the MOI, viral replication in the cells which permitted viral uptake, but without viral spreading due to the lack of receptor. Nonetheless, there was sufficient uptake and replication to result in a roughly 5000 fold increase of viral RNA over input RNA from the virus added to the culture. RNASeq was conducted on the cell lysates as a secondary confirmation of inhibition of viral replication as demonstrated in FIG. 7B. These data demonstrate that VPA, DHA and VPA+DHA each have inhibition of viral replication. However, the degree of inhibition may be underestimated due to the small amount of viral RNA detectable. These data demonstrate that despite the lack of ACE2 (which is known to be downregulated by VPA), VPA as well as the combination of VPA+DHA inhibit viral replication and provide an antiviral strategy against coronaviruses.

Discussion. The coronavirus (e.g., SARS-CoV2) pandemic is one of the most impactful infections in modern history. While a historic number of people have been infected and died from this disease, the worldwide response to vaccine development has been equally impressive. Unfortunately, incomplete vaccination and persistent viral mutations have hampered attempts to eradicate this disease, and ultimately vaccination therapy alone is not a viable long-term strategy. Broad-spectrum antiviral drug development must be pursued and optimized to combat this virus, its variants, and other similar highly transmissible respiratory viruses. Early bioinformatic methodologies greatly facilitated strategies for antiviral drug development, which demonstrated the important pathways of interaction between SARS-CoV2 proteins and cellular proteins. Among the early targets identified by this methodology was HDAC2 (Tomazou, M., et al., Brief Bioinform, 2021. 22(6)), found to interact with nsp5 (Gordon, D. E., et al., Nature, 2020. 583(7816): p. 459-468). Efforts have been underway to evaluate the efficacy of HDAC inhibitors on coronavirus replication (Teodori, L., et al., Front Pharmacol, 2020. 11: p. 582003; and Liu, K., et al., ACS Pharmacol Transl Sci, 2020. 3(6): p. 1361-1370). Among the drugs studied was the short-chain fatty acid valproic acid (Koch-Weser, J. and T. R. Browne, N Engl J Med, 1980. 302(12): p. 661-6), a commonly used antiseizure agent also used for bipolar disorder (Hsieh, T. C., et al., J Psychiatr Res, 2022. 149: p. 339-343; and Kowatch, R. A., et al., J Child Adolesc Psychopharmacol, 2015. 25(4): p. 306-13) and migraine headaches. VPA was first approved in the 1970s and has a well-established safety profile and therapeutic index. While there are well-known toxicities, including fetal teratogenicity, somnolence, thrombocytopenia, and rare episodes of hepatitis and pancreatitis, it has been well tolerated by most patients over prolonged periods.

The most significant concern with VPA is teratogenicity which can cause an increase in the risk of neural tube defects in the fetuses of pregnant women (Kultima, K., et al., Environ Health Perspect, 2004. 112(12): p. 1225-35). This activity may be related to the HDAC2 inhibitory activity of VPA (Kramer, O. H., et al., EMBO J, 2003. 22(13): p. 3411-20). Early in vitro testing of valproic acid against SARS-CoV2 (Gordon, D. E., et al., Nature, 2020. 583(7816): p. 459-468) indicated no measurable inhibition of viral infection. In retrospect, however, these studies were performed in a dose range of VPA (M range) significantly less than the IC₅₀ of HDAC2 inhibition (Kramer, O. H., et al., EMBO J, 2003. 22(13): p. 3411-20) (mM range). In vitro studies of VPA in the mM range have demonstrated that treatment of cells reduces expression of the SARS-CoV2 receptor ACE2 as well as IL-6 and ICAM-1 (Singh, S., International Journal of Respiratory and Pulmonary Medicine, 2020. 7(3)), although the high dose requirements in high-throughput viral replication assays hindered enthusiasm for clinical use. High throughput screening assays, however, are developed to identify agents which inhibit SARS-CoV2 replication directly. HDAC inhibition is an indirect strategy. These agents must first inhibit histone deacetylation to alter gene expression in susceptible gene loci. Induction of altered gene expression takes at least 24 hours (Kramer, O. H., et al., EMBO J, 2003. 22(13): p. 3411-20), as demonstrated in FIG. 2. In many SARS-CoV2 assays, viral replication is completed within 24 hours.

Serum VPA levels were available for 442 of the 691 patients during the study period, and when corrected for the seizure disorder therapeutic range, the calculated OR for contracting COVID-19 was 0.218 in the therapeutic VPA cohort. While these dramatic reductions were statistically significant, the clinical relevance of these findings was lessened by the small number of patients in the study. Similarly, Collazos et al. found in a retrospective study of 165 VPA-treated patients admitted to the hospital for COVID-19 that the VPA-treated patients had a shorter duration of symptoms, lower in-hospital respiratory worsening, lower ICU admissions, and fewer pulmonary infiltrates than comparable controls matched for sex, age, and date of admission (Collazos, J., et al., PLoS One, 2022. 17(1): p. e0262777). The mortality, however, was not statistically different. Like before, the small number of patients made interpretation difficult. Disclosed herein are the results of a large cohort study of the association of VPA with COVID-19 that showed a significant association of VPA use with diminished disease contraction and severity. Using a national database from Optum, patients prescribed VPA have a 25% decreased risk of contracting COVID-19 in an exact-matched multivariate analysis determined by nucleic acid testing. Since the dose required for antiviral activity is relatively high (>85 μg/mL, or 0.6 mM) in the in vitro testing, such a protective effect is impressive as fewer than 25% of VPA-treated patients are in the predicted optimal therapeutic range for antiviral activity.

Once a putative protective effect was demonstrated, the next set of experiments were designed to understand why this activity, was not demonstrated in prior in vitro viral replication assays. The results show that the IC₅₀ for VPA in a conventional coronavirus replication assay (HCoV-229E was used as a prototype), was nearly 5 mM, which translates to over 720 μg/mL, nearly six times above the toxicity level in humans (125 μg/mL). The dose required in these assays was considered toxic, and no further evaluation would have been recommended. However, HDAC inhibitors work primarily by altering transcription in a process that often takes >24 hours. Thus, the protective effect of VPA in preventing conversion to COVID-19 positive may have been a manifestation of chronicity of VPA administration, in which gene induction occurs at a lower dose of VPA as previously identified in cancer therapeutic models (Xia, Q., et al., Cancer Res, 2006. 66(14): p. 7237-44). Time course experiments were performed in coronavirus permissive cell lines and the results demonstrated that gene expression profile changes require at least 24 hours before they can be detected, and in some cases, they are maximal between 72-96 hours (FIG. 2). A comparison of the SARS-CoV2 interacting genes demonstrates that the expression of multiple genes was reduced, involving at least four SARS-CoV2 pathways (FIGS. 2B and 2C). Incubation of the HCoV-229E permissive cell line MRC5 with valproic acid for at least 24 hours prior to infection demonstrated that the IC₅₀ for inhibition of viral replication was reduced by nearly 10-fold, down to roughly 0.6 mM (corresponding to 85 μg/mL serum levels). Significantly, in vitro drug concentrations do not necessarily correlate precisely with serum drug levels, as serum drug delivery is a far more complex process with drug partitioning into different functional compartments and dynamically changing equilibrium of free drug based on the tightly regulated carrier proteins. Nonetheless, the in vitro results are consistent with the 25% reduction in COVID-19 positive testing among patients on active valproic acid treatment, as less than 25% of patients have VPA levels more than 85 μg/mL. To lessen clinical toxicity it was tested whether the activity of VPA could be enhanced by other methods.

It was assessed whether the combination of PUFA DHA and VPA in vitro alters the antiviral activity of VPA. Surprisingly, the combination enhanced the antiviral activity, reducing the dose requirement of VPA to a level of 0.1 mM, a dose easily achievable in human patients without the side effects most commonly associated with the higher doses required for antiseizure activity (FIG. 4B). DHA did not have any HDAC inhibitory activity but did have antiviral activity.

The combination of valproic acid and DHA potently inhibits coronavirus replication of both HCoV-229E and SARS-CoV2. The combination of DHA with other HDAC inhibitors demonstrates that enhanced activity is maintained with other HDAC2 inhibitors. Additionally, the gene expression profiles of MRC5 cells treated with VPA, DHA, or VPA+DHA both in the presence or absence of coronavirus HCoV-229E demonstrate that the additional antiviral activity is not a consequence of further HDAC inhibition. Instead, it appears that the combination activates pathways involved in interferon and downstream related genes with a scatterplot demonstrating a profound change in the overall gene expression profile to inhibit viral replication. Hence, this combination represents a target for coronavirus antiviral therapy and a new class of antiviral combination therapies.

Linoleic acid (LA) is an omega-6 polyunsaturated fatty acid (PUFA). It is also considered an essential fatty acid as it can give rise to multiple other fatty acid metabolites, including the omega-3 fatty acids- alpha-linoleic acid (ALA), eicosapentaenoic acid (EPA), and docosahexaenoic acid (DHA). Toelzer et al. demonstrated that the receptor binding protein (S, or Spike) of SARS-CoV2 has a hydrophobic pocket capable of sequestering linoleic acid (LA), causing a conformational change impacting viral infectivity (Hsieh, T. C., et al., J Psychiatr Res, 2022. 149: p. 339-343). Combining LA with remdesivir resulted in a significant improvement in the inhibition of coronavirus replication. The combination of LA with VPA did not have a similar effect in HCoV-229E; however, sequence comparison of the binding region between SARS-CoV2 and HCoV-229E predicted a lack of LA binding in HCoV-229E. Furthermore, MRC5 lacks the ACE2 receptor (Uemura, K., et al., Sci Rep, 2021. 11(1): p. 5376), preventing testing with SARS-CoV2 in MRC5 cells.

Additionally, HCoV-229E uses the human aminopeptidase N as its receptor (Yeager, C. L., et al., Nature, 1992. 357(6377): p. 420-2), so the LA lipid binding region identified by Toelzer et al. may not be a universally applicable strategy for all coronaviruses. Combining VPA with the three primary omega-3 fatty acids (ALA, DHA, EPA) demonstrates that the selectivity of the combination favors DHA. While LA may help inhibit SARS-CoV2 by directly affecting viral conformation, DHA causes gene expression changes, providing a broader strategy as it is not as susceptible to COVID-19 variants such as D614G, lacking the susceptibility to LA binding (Sofia, F. O. A., et al., Comput Struct Biotechnol J, 2022. 20: p. 139-147).

In summary, the epidemiological data indicate that patients who take VPA have a decreased rate of COVID-19 test positivity, a decreased rate of ER visits, a decreased rate of hospitalization if they contract COVID-19, a decreased rate of ICU admissions, and a decreased rate of mechanical ventilation. Given the time window during which this data was extracted, this effect continued through the transition from the alpha to the delta variant, indicating an overall effect not easily lost with virulence mutations. Serum VPA testing nationally indicates that about 25% of the patients who take VPA would have had sufficient serum levels for inhibition of SARS-COV2, according to the in vitro data. However, these associations do not show causality. The small branched-chain fatty acid VPA combined with the omega-3 PUFA DHA results in a marked antiviral activity against coronaviruses. This treatment appears effective due to activating a large set of antiviral genes involving the re-activation of innate immune genes, including many interferon-regulated genes. This strategy is attractive as it may retain efficacy even with the emergence of mutant coronavirus variants while minimizing disease severity and spread.

What is claimed is:

1. A method of treating a subject having a coronavirus infection, the method comprising administering to the subject one or more therapeutically effective doses of valproic acid and docosahexaenoic acid.

2. The method of claim 1, wherein valproic acid and docosahexaenoic acid inhibit replication of the coronavirus.

3. A method of treating or preventing COVID-19 in a subject, the method comprising administering to the subject one or more therapeutically effective doses of valproic acid and docosahexaenoic acid.

4. A method of preventing or inhibiting a coronavirus infection in a subject, the method comprising administering to the subject one or more therapeutically effective doses of valproic acid and docosahexaenoic acid.

5. A method of inhibiting replication of a coronavirus in a subject having a coronavirus infection, the method comprising administering to the subject one or more therapeutically effective doses of valproic acid and docosahexaenoic acid.

6. The method of claim 5, wherein valproic acid and docosahexaenoic acid inhibit replication of the coronavirus.

7. A method of reducing one or more symptoms of a coronavirus infection or COVID-19 in a subject, the method comprising administering to the subject one or more therapeutically effective doses of valproic acid and docosahexaenoic acid.

8. The method of claim 7, wherein the one or more symptoms the coronavirus infection or COVID-19 are fever, sore throat, malaise, difficulty breathing, fatigue, muscle aches, or difficulty with cognition or concentration.

9. A method of inhibiting replication of a coronavirus in a cell, the method comprising contacting the cell infected with the coronavirus with one or more therapeutically effective doses of valproic acid and docosahexaenoic acid.

10. The method of any of the preceding claims, wherein the one or more therapeutically effective doses of valproic acid and docosahexaenoic acid are administered in an additive or a synergistic combination.

11. The method of any of the preceding claims, further comprising administering to the subject one or more therapeutically effective doses of remdesivir.

12. The method of any of the preceding claims, wherein the valproic acid or docosahexaenoic acid is administered in a composition comprising at least one pharmaceutically acceptable carrier, diluent or excipient.

13. A method of treating a subject having a coronavirus infection, the method comprising administering to the subject one or more therapeutically effective doses of (a) depsipeptide and (b) docosahexaenoic acid.

14. The method of claim 13, wherein the (a) depsipeptide and (b) docosahexaenoic acid inhibit replication of the coronavirus.

15. A method of treating or preventing COVID-19 in a subject, the method comprising administering to the subject one or more therapeutically effective doses of (a) depsipeptide and (b) docosahexaenoic acid.

16. A method of preventing or inhibiting a coronavirus infection in a subject, the method comprising administering to the subject one or more therapeutically effective doses of (a) depsipeptide and (b) docosahexaenoic acid.

17. A method of inhibiting replication of a coronavirus in a subject having a coronavirus infection, the method comprising administering to the subject one or more therapeutically effective doses of (a) depsipeptide and (b) docosahexaenoic acid.

18. The method of claim 17, wherein (a) depsipeptide and docosahexaenoic acid inhibit replication of the coronavirus.

19. A method of reducing one or more symptoms of a coronavirus infection or COVID-19 in a subject, the method comprising administering to the subject one or more therapeutically effective doses of (a) depsipeptide and (b) docosahexaenoic acid.

20. The method of claim 19, wherein the one or more symptoms the coronavirus infection or COVID-19 are fever, sore throat, malaise, difficulty breathing, fatigue, muscle aches, or difficulty with cognition or concentration.

21. A method of inhibiting replication of a coronavirus in a cell, the method comprising contacting the cell infected with the coronavirus with one or more therapeutically effective doses of (a) depsipeptide and (b) docosahexaenoic acid.

22. The method of any of the preceding claims, wherein the one or more therapeutically effective doses of (a) depsipeptide and (b) docosahexaenoic acid are administered in an additive or a synergistic combination.

23. The method of any of the preceding claims, wherein the (a) depsipeptide or (b) docosahexaenoic acid is administered in a composition comprising at least one pharmaceutically acceptable carrier, diluent or excipient.

24. The method of any of the preceding claims, wherein the administration is systemic (intravenous), oral, or a combination thereof.

25. The method of any of the preceding claims, wherein the subject is infected or has previously been infected with a coronavirus.

26. The method of any of the preceding claims, wherein the coronavirus is SARS-CoV, MERS-CoV, SARS-CoV-2, HCoV 229E, HCoV NL63, HCoV OC43, HCoV HKU1, D614G, B.1.1.7, 20I/501Y.V1, B.1.351 or 20H/501.V2, P.1, 20J/501Y.V3, 20C/S:452R, Cluster 5 Variant, XBB.1.5, XBB.1.5.70, XBB.1.5.68, XBB.1.5.72, XBB.1.5.10, XBB.1.5.59, XBB.1.5.1, XBB.1.16, XBB.1.16.6, XBB.1.16.11, XBB.1.16.15, XBB.1.6.1, EG.5, XBB.1.9, XBB.1.9.1, XBB.1.9.2, XBB.2.3, BA.1, BA.5, BA.2.86, HV.1, FL.1.5.1, HK.3, JD.1.1, JF.1, GK.1.1., HF.1, BA.2, BA.2.12.1, BA.2.12.2, BA.4, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1, or BQ.1.1.

27. The method of any of the preceding claims, wherein the valproic acid total dose per day is independently selected upon each occurrence from about 500 mg to about 2000 mg.

28. The method of any of the preceding claims, wherein the docosahexaenoic acid total dose per day is independently selected upon each occurrence from about 500 mg to about 1000 mg.

29. The method of any of the preceding claims, wherein the depsipeptide total dose per day is independently selected upon each occurrence from about 1 mg/m² to 10 mg/m².

30. The method of claim 10, wherein the one or more therapeutically effective doses of valproic acid and docosahexaenoic acid are in a ratio of 2.5:1 to 5:1.

31. The method of claim 22, wherein the one or more therapeutically effective doses of depsipeptide is 1 mg/mg² to about 10 mg/m² and the one or more therapeutically effective doses of docosahexaenoic acid is 25 μM or 500 mg to 1,000 mg/day.

32. A method of inhibiting, treating or preventing a coronavirus infection in a subject, the method comprising administering to the subject having said infection a plurality of therapeutically effective doses of valproic acid and docosahexaenoic acid.

33. The method of claim 32, wherein the plurality of therapeutically effective doses of valproic acid and docosahexaenoic acid is one or more doses administered per day for two or more days per week.

34. A method of inhibiting, treating or preventing a coronavirus infection in a subject, the method comprising administering to the subject having said infection a plurality of therapeutically effective doses of (a) depsipeptide and (b) docosahexaenoic acid.

35. The method of claim 34, wherein the plurality of therapeutically effective doses of (a) depsipeptide and (b) docosahexaenoic acid is one or more doses administered per day for two or more days per week.

36. The method of claim 33 or 34, wherein dosing is continued for one or more weeks per month.

37. The method of claim 36, wherein dosing is continued for one or more months per year.

38. The method of claim 32 or 34, wherein the coronavirus is pathogenic to humans.

39. The method of claim 32 or 34, wherein the coronavirus is SARS-CoV, MERS-CoV, SARS-CoV-2, HCoV 229E, HCoV NL63, HCoV OC43, HCoV HKU1, D614G, B.1.1.7, 20I/501Y.V1, B.1.351 or 20H/501.V2, P.1, 20J/501Y.V3, 20C/S:452R, Cluster 5 Variant, XBB.1.5, XBB.1.5.70, XBB.1.5.68, XBB.1.5.72, XBB.1.5.10, XBB.1.5.59, XBB.1.5.1, XBB.1.16, XBB.1.16.6, XBB.1.16.11, XBB.1.16.15, XBB.1.6.1, EG.5, XBB.1.9, XBB.1.9.1, XBB.1.9.2, XBB.2.3, BA.1, BA.5, BA.2.86, HV.1, FL.1.5.1, HK.3, JD.1.1, JF.1, GK.1.1., HF.1, BA.2, BA.2.12.1, BA.2.12.2, BA.4, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1, or BQ.1.1.

40. The method of claim 32 or 34, wherein the administration is oral, intravenous, or a combination thereof.

41. The method of claim 32 or 34, further comprising administering a therapeutic effective amount of remdesivir to the subject.

42. The method of claim 32, wherein the valproic acid and docosahexaenoic acid is administered to the subject immediately after infection or any time within one day to 5 days after infection or at the earliest time after diagnosis of infection with the coronavirus.

43. The method of claim 32, wherein the valproic acid and docosahexaenoic acid are administered to the subject as a primary antiviral therapy, adjunct antiviral therapy, or a co-antiviral therapy, or wherein the administration comprises separate administration or coadministration of valproic acid and docosahexaenoic acid with at least one other antiviral composition or with at least one other composition for treating one or more symptoms associated with said coronavirus infection.

44. The method of claim 34, wherein the (a) depsipeptide and (b) docosahexaenoic acid is administered to the subject immediately after infection or any time within one day to 5 days after infection or at the earliest time after diagnosis of infection with the coronavirus.

45. The method of claim 34, wherein the (a) depsipeptide and (b) docosahexaenoic acid are administered to the subject as a primary antiviral therapy, adjunct antiviral therapy, or a co-antiviral therapy, or wherein the administration comprises separate administration or coadministration of depsipeptide and docosahexaenoic acid with at least one other antiviral composition or with at least one other composition for treating one or more symptoms associated with said coronavirus infection.

46. A kit for use in treating a subject suffering from a coronavirus infection, said kit comprising: (a) valproic acid; and (b) docosahexaenoic acid.

47. A kit for use in treating a subject suffering from a coronavirus infection, said kit comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone.

48. A kit for use in treating a subject suffering from a coronavirus infection, said kit comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

49. A kit for use in preventing or inhibiting a coronavirus infection in a subject, said kit comprising: (a) valproic acid; and (b) docosahexaenoic acid.

50. A kit for use in preventing or inhibiting a coronavirus infection in a subject, said kit comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone.

51. A kit for use in preventing or inhibiting a coronavirus infection in a subject, said kit comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

52. A kit for use in inhibiting replication of a coronavirus infection in a subject, said kit comprising: (a) valproic acid; and (b) docosahexaenoic acid.

53. A kit for use in inhibiting replication of a coronavirus infection in a subject, said kit comprising: (a) valproic acid; (b) docosahexaenoic acid; and (c) molnupiravir, 4′-fluorouridine, favipiravir, remdesivir, nirmatrelvir, ritonavir, a combination of nirmatrelvir and ritonavir, GC-376, cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, fluvoxamine, or prednisone.

54. A kit for use in inhibiting replication of a coronavirus infection in a subject, said kit comprising: (a) valproic acid; (b) docosahexaenoic acid; and (c) remdesivir, Nafamostat, favilavir, bamlanivimab, Olumiant and Baricinix (baricitinib), hydroxychloroquine/chloroquine, Casirivimab, imdevimab, PTC299, Leronlimab, Bamlanivimab, Lenzilumab, Ivermectin, aviptadil, Metformin, AT-527, tocilizumab, niclosamide, convalescent plasma, famotidine, lopinavir-ritonavir, infliximab, AZD7442, AZD7442, CT-P59, Heparin (UF and LMW), VIR-7831 (GSK4182136), JS016, sarilumab, SACCOVID (CD24Fc), adalimumab, COVI-GUARD (STI-1499), Dexamethasone, PB1046, Galidesivir, Bucillamine, PF-00835321 (PF-07304814), Eliquis (Apixaban), lanadelumab, hydrocortisone, canakinumab, Colchicine, BLD-2660, favilavir/avifavir, Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, acalabrutinib, captopril, losartan, atorvastatin, simvastatin, camostat, nafamostat, macrolides, clindamycin, doxycycline, ivermectin, niclosamide, amiodarone, verapamil, tranexamic acid, chlorpromazine, umifenovir, oseltamivir, linagliptin, baricitinib, sulfated glycosaminoglycans (UFH and LMWHs), DAS181, rhACE2, REGN10933, or REGN10987.

55. The kit of claims 46-54, further comprising at least one pharmaceutically acceptable carrier, diluent or excipient.

56. The kit of claim 46-54, further comprising instructions for using valproic acid and docosahexaenoic acid in treating a coronavirus infection.

57. The method of any of the preceding claims, further comprising administering to the subject one or more therapeutically effective doses of a polymerase inhibitor.

58. The method of claim 57, wherein the polymerase inhibitor is molnupiravir, 4′-fluorouridine, favipiravir, or remdesivir.

59. The method of claim 57 or 58, wherein the therapeutically effective doses of valproic acid and docosahexaenoic acid are administered in an additive or synergistic combination.

60. The method of any of the preceding claims, further comprising administering to the subject one or more therapeutically effective doses of a protease inhibitor.

61. The method of claim 60, wherein the protease inhibitor is nirmatrelvir, ritonavir, a combination of nirmatrelvir and ritonavir, or GC-376.

62. The method of any of the preceding claims, further comprising administering to the subject one or more therapeutically effective doses of a helicase inhibitor.

63. The method of claim 62, wherein the helicase inhibitor is cepharanthine, cefoperazone, dihydroergotamine, cefpiramide, ergoloid, ergotamine, netupitant, DPNH (NADH), lifitegrast, nilotinib, tubocurarin, lumacraftor, emend, irinotecan, enjuvia, zelboraf, cromolyn, diosmin, Risperdal, or differin.

64. The method of any of the preceding claims, further comprising administering to the subject one or more therapeutically effective doses of an inhibitor of host proteins supporting viral replication.

65. The method of claim 64, wherein the inhibitor of host proteins supporting viral replication is plitidepsin.

66. The method of any of the preceding claims, further comprising administering to the subject one or more therapeutically effective doses of a non-vaccine biologic.

67. The method of claim 66, wherein the non-vaccine biologic is convalescent plasma, actemra, a monoclonal antibody specific for a viral protein.

68. The method of any of the preceding claims, further comprising administering to the subject one or more therapeutically effective doses of an inhibitor of viral attachment and entry.

69. The method of claim 68, wherein the inhibitor of viral attachment and entry is human recombinant soluble angiotensin converting enzyme-2 (ACE2) or camostate mesylate and analogs thereof.

70. The method of any of the preceding claims, further comprising administering to the subject one or more therapeutically effective doses of a selective serotonin reuptake inhibitor.

71. The method of claim 70, wherein the selective serotonin reuptake inhibitor is fluvoxamine.

72. The method of any of the preceding claims, wherein the subject does not have a seizure disorder or bipolar disorder.


SEQUENCE LISTING (1 sequence)

SEQ ID NO: 1 - DNA, 39 bp, synthetic construct
  source 1..39: mol_type = other DNA
ccactctcaacagcaaatacattttctgaataaccaaca